(19) United States
(12) Patent Application Publication
(10) Pub. No.: US 2003/0125609 A1
(43) Pub. Date: Jul. 3, 2003

(54) Method for reliable measurement in medical care and patient self monitoring

(76) Inventor: Robert Becker, Carrabassett Valley, ME (US)

Correspondence Address: Cummings & Lockwood LLC, Granite Square, 700 State Street, P.O. Box 1960, New Haven, CT, 06509-1960, US

(21) Appl. No.: 10/213,305

(22) Filed: Aug. 5, 2002

Related U.S. Application Data
(60) Provisional application No. 60/310,058, filed on Aug. 3, 2001.
(60) Provisional application No. 60/391,492, filed on Jun. 25, 2002.

(51) Int. Cl. C12Q 1/00; G06F 19/00; G01N 33/48; G01N 33/50; A61B 5/00

(52) U.S. Cl. 600/300; 435/4; 702/19

(57) ABSTRACT

Using the method of invention makers of devices, tests, methods of assessment, software programming or hardware programming or other procedures for determining the health or health related status of a person can reach sufficient reliability in the measurements provided to the user that the true status of an individual can be described and the course of the measurements interpreted meaningfully as part of a physician or patient or member of the public seeking well-being and health adopting a prescribed or self prescribed plan for monitoring health and achieving desired health goals. Computer software or hardware programming for these methods and for methods disclosed in the co-pending provisional patent applications hereby expressly incorporated above and below by reference as part of the present disclosure enables the physician, health professional, patient, or healthy user to establish reliability in measurements in examinations and tests. Health and disease management applications are to provide accurate interpretations of health indicators based in the improved precision of measurement, earlier detection of changes in health status for both health monitoring and disease management, to provide statistically grounded evidence for possible causal relations among health interventions, disease processes and the clinical or health status of the person.

### CROSS-REFERENCE TO RELATED PRIORITY APPLICATIONS

This patent application claims priority on the present inventor's following co-pending provisional patent applications which are each hereby expressly incorporated by reference as part of the present disclosure: serial No. 60/258,262, filed Dec. 26, 2000, entitled “Method of Administering ChEIs for treating Alzheimer's Disease”; serial No. 60/274,981, filed Mar. 12, 2001, entitled “Method of Drug Development for Selective Use with Individual, Treatment Responsive, Patients;” serial No. 60/301,526, filed Jun. 28, 2001, entitled ”Method of Drug Development for Selective Use with Individual, Treatment Responsive, Patients and the Applications of the Method of Drug Development in Medical Care;” serial No. 60/310,058, filed Aug. 3, 2001; entitled “Method of Reliable Measurement in Medical Care and Patient Self Monitoring; international application no PCT/US01/49457, filed Dec. 26, 2001; and “Method for Reliable Measurement in Medical Care and Patient Self Monitoring,” serial No. 60/391,492, filed Jun. 25, 2002.

### FIELD AND OVERVIEW OF THE INVENTION

The present invention is directed to a method of using statistical and scientific knowledge and theory to provide reliable assessments of health status as indicated by commonly employed measures of health and illness, medical tests, scales whether self administered or administered to the individual being tested, medical or other human activity monitoring instruments, or any other form of health related assessment. The present invention differs from current practice: current health related and professional medical assessment methods do not establish the error components of measurement for the subject and do not develop a plan of assessment out of these reliability studies. Current methods do not provide for a plan of assessment with sufficient reliability to optimally use the information from assessment as indications of the patient's or user's actual progress towards health goals. In our medical and personal health applications of scientific and medical scales, tests and examinations we lose information because we depend on personal and professional judgments to interpret how correctly the tests, scales, examinations, measure the true condition of the subject. The present invention specifically addresses each of these deficiencies in current methods of self and medical, health or treatment monitoring.

The method of invention in applications to self-care and medical care differs from current practices. Currently we disseminate health information and new medical findings in articles, publications, broadcasts, advertisements, and so forth. This does not integrate the new findings and their normative, health engendering, or therapeutic implications with the applications to individual patients or by a person in his or her own life. The user, reader or listener must interpret the meaning, implications, and methods of application of research for his or her own health and for the health of others. The method of invention overcomes this need for personal or professional judgments to interpret new standards of health and treatment for use with individuals. Under the method of invention new findings can be integrated by providers, developers or manufacturers of measures of health and illness, medical tests, scales whether self administered or administered to the individual being tested, medical or other human activity monitoring instruments, or any other form of health related assessment such that the medical information becomes grounds for interpretation of the reliable assessments of health status. It is anticipated that this information transfer will be ongoing; a new article or other announcement of a medical advance will be provided to the user in a form that integrates the new information into the device or system for interpretation so the patient will have a more direct, personally relevant, specific interpretation of the latest medical and health information in terms of the import for the patient his or her self.

The method of the invention develops a model that tests the reliability of any medical or health assessment, takes into account the health or medical goals of use, develops a plan of assessment adequate to provide the reliability required for the assessments to be useful indicators of progress towards health goals, provides a display or output that interprets the current measurements from examinations, tests, scales, instruments, methods, systems, in relation to the patient or user aims, provides a program or processing system that interprets the status indicated by the processed assessments, and updates the interpretive database with new medical or health advances.

The method of invention establishes the reliability of a health or clinical measurement as an indicator of the person's true condition by determining the error component of measurement. The error component of measurement can be expressed as a confidence interval of measurement (CIm) with a specific probability by multiplying the standard error of measurement for the data set developed by the user of a test, scale or examination by the required amount for the resulting interval to contain on average the percentage of observations implied by the specific probability. For example, using repeated measures taken under conditions free of systematic influences a 95% CIm is derived from the error of measurement multiplied such that 19 out of 20 measurements taken fall within the resulting confidence interval of measurement.

The method of invention develops confidence intervals of measurement for health and disease uses of clinical examinations, tests, scales, or other measurements and compares the adequacy of the reliabilities of single and combinations of multiple administrations of the examination, test, scale or other measurements to select a measure with adequate reliability to achieve the clinical or health monitoring purposes of the user.

The method of measurement uses the reliabilities of measurement expressed in confidence intervals of measurement or otherwise as needed for the application to distinguish statistically significant deviations from a projected health or clinical course measured by the indicator, to provide probabilities for any deviations from the earlier course in latter testing, and to support the clinical or health interpretation of the changes or lack thereof in measurement.

The method of invention uses confidence intervals of measurement and the above mentioned methods of reliably detecting deviations from a predicted course as test criteria for hypothesis testing in an n-of-1 trial. Consequently, n-of-1 trials become more practically available to health professionals and the public to evaluate health and disease interventions.

The method of invention uses a calculated measure of informativeness defined as the reduction of uncertainty associated with the information becoming available to compare the adequacy of the different methods of processing measurements, of designing research, experimental, or observational studies, clinical trial designs, for the clinical purposes or health purposes or aims of the health professionals in patient care or well-being or health activities of individuals.

The method of invention develops a Disease Management Plan, Health or Clinical Course Monitoring plan to guide health care decision making.

The method of invention uses software programming or hardware design for a computer, data processing device, or other device to make these methods available to users.

Using these resources the individual pursuing health goals or the professional health care provider can quickly detect effects on health status from interventions or changes in health habits or practices and can gather evidence of the importance of the intervention or changed practice to health status changes.

As may be recognized by those skilled in the pertinent art on the teachings herein, the method of the present invention is applicable to health and disease management, the development, registration and use of any measures of health and illness, medical tests, scales, examinations, whether self administered or administered to the individual being tested, medical or other human activity monitoring instruments, or any other form of health related assessment where without the benefits of the method of invention the user must rely on clinical or health judgments to interpret the precision and health or treatment implications of an assessment. The invention provides scientific and statistical research based grounds to self-care health and medical evaluations, assessments, decision making and treatment. The invention, in new applications to individuals for health and disease monitoring, uses statistical and scientific arts widely practiced to study groups of patients and for bio-medical research.

### BACKGROUND OF THE INVENTION

Each individual, and each physician, must assume that his or her methods of personal health assessment or professional clinical methods of assessment have sufficient reliability and validity—are sufficiently free from random or systematic error from one administration to another and express the actual or true condition of the person. Any rational system of decision making is only as strong as its weakest link. When judgments about personal health, or a physician's clinical judgments, are grounded in unreliable or inexact measurements the conclusions and decisions lose validity. Self-care for health and physicians' medical care of patients must be grounded in reliable and valid individualized assessment of an individual's health status and clinical response to disease or treatments. The method of invention provides statistical and scientific grounds for personal, physicians' and other health care providers', and for health care service or funding organizations' decision making in areas where now in self-care, medical care, and health and disease management and funding for health services, or other health related services we depend upon the 37 unsystematic” clinical experiences and judgments of professionals and the personal judgments of individuals providing self-care. (Guyatt et al., 2000)

We illustrated the problem of how a state of the art medical assessment used by trained experts may not provide sufficiently reliable measurements of the patient's true condition to be grounds for health care decisions. Yet physicians use these methods of medical assessments for medical care decisions, and persons in self-care use similar methods, without controls for the errors in measurement. Becker and Markwell (2000) show the error in the tests used to assess the cognitive status of Alzheimer's disease (AD) patients is sufficiently large to obscure both the short term decline in cognitive performance typical of the disease and treatment effects. This leaves the patient and the practicing physician no reliable clinical assessments of individual patients to inform clinical judgments of probable future status or the effects from treatment interventions. In many health and medical conditions the methods of assessment have unknown reliability. An assessment, test, scale, or examination used without taking into account the necessary conditions to assure reliability is an imprecise indicator of current health status, changes, or effects from changes in health habits, practices or treatments. Weights, blood pressures, blood glucose assays, exercise measures, physical performance assessments, scales for mood or cognition or other bodily states, like all measurements have both systematic and random errors that make the measure of unknown precision. For decision making to reach a given level of certainty the elements that go into the decision making must each have sufficient precision, accuracy, certainty or reliability such that the decision choice can be depended upon as a true indicator for the purposes for which it is intended.

The error variance in the repeated uses with a person of medicine's clinical examination methods and laboratory procedures, or in personal use of home monitoring or personal methods of health assessment, is not studied scientifically and statistically and the effects of error variance taken into account in each assessment. The method of this invention enables the individual providing self-care, or the physician, or others, to conduct and interpret assessments such that the error component of measurement is taken into account and a course of assessments over time becomes a more precise predicator for the individual or physician to rely on for health care decisions.

At one end of a spectrum of reliability, we have no personal or clinical methods of assessment of the patient sufficiently free from error to reliably distinguish changes over short periods of time or changes from treatment from random test error. (Becker and Markwell, 2000) On the other end of the spectrum of reliability even medicine's most reliable assessments—for example laboratory examinations—offer an interpretation based on a normal range of test results which allow 5% (or thereabouts) of all routine observations to be classified as outside the normal range. When the use of health and medical assessments does not integrate a model that takes account of this variable error range among outcome measures both the individual in self care and the practicing physician must resort to personal judgments and guesses about the accuracy of the information on which decisions will be based.

The n-of-1 trial provides an illustration of the limitations imposed by assessments of unknown reliability. The n-of-1 trial is a method of randomly and blindly assigning treatment and placebo in one individual to ascertain whether the intervention provides a benefit. It is a scientific and statistical design to provide a gold standard for the question any individual interested in personal health asks—“Does this health practice benefit me?” (Guyatt et al., 2000; Larson et al., 1993; Backman and Harris, 1999) Assessments of an individual obtained under blind conditions of sequential treatment by active treatment and placebo are compared to determine the efficacy or safety of the treatment in the individual patient. However, the n-of-1 trial has limitations: the randomization procedure is time consuming; the trial exposes the patient to periods of no treatment in placebo treatment; the trial often has less statistical power than a clinical trial increasing the likelihood of erroneously continuing or discontinuing a treatment on the basis of the n-of-1 trial results or the results being inconclusive. Therefore the clinician will not want to use the n-of-1 trial technique when its use can be avoided. (Johannessen and Fosstveldt, 1991) One source of limitation is that the current n-of-1 trial methods do not call for the precision of measures for the individual to be established. The n-of-1 trial now uses methods to control error of measurement effects that are used in group comparisons in randomized controlled trials. In randomized controlled trials error of measurement is taken into account by comparing the means of measurements in different patients with the assumption that the random errors of measurement have a zero or equivalent difference in their contributions to the means used for comparisons. Establishing the error of measurement and developing a plan of assessment based on the limitation of measurement due to error and the uses of the measurements make n-of-1 trials more practical. Multiple exposures of the person to the different conditions no longer are needed. Error is controlled not by averaging responses from multiple exposures to a treatment condition but by determining the precision of measurement used with the individual in the n-of-1 trial. Thus a statistically significant deviation from the expected course after a change in treatment condition becomes evidence with known statistical strength that effects may follow from the change in treatment conditions.

Using the method of invention the n-of-1 trial becomes a model more practically available to any individual to evaluate the efficacy, or safety, of a health practice or intervention for the individual personally. Without the method of invention and its uses of confidence intervals of measurement, criteria of clinical significance, criteria of statistical significance, (see reference above and descriptions below in methods) decisions must be based on less precise assessments and interpretations of less precise assessments. Measurements with established precision cannot replace personal judgments by the individual engaged in self care or clinical judgment by a physician. Measurements of known precision can better ground all forms of judgment and more directly interact with health care and medical research to provide more exact or accurate interpretations and predications for an individual. The inference that a health care practice or medical treatment applies to a person or benefits a person today depends on the physician's unsystematic clinical experiences and unsystematic clinical judgment which has unclear or no scientific evidentiary support. (Guyett et al., 2000) The individual engaged in self-care can at best be expected to reach the unsystematic reliability available to physicians. The method of invention replaces unsystematic experience with statistically and scientifically reliable derived evidence of precision in health and clinical measurements.

### THE SUMMARY OF THE INVENTION

The method of the present invention recognizes, and corrects, the current inability of the individual or clinician to manage the health care of the individual in self or clinical practice systematically and rationally because of the undetermined precision in methods of clinical and self assessment. The methods of invention provide a device(s) or system(s) or combined device(s) and system(s) that the consumer or health professional may use as a tool in personal or professional health care decision making.

Preferred methods of statistical and scientific analysis of precision and reliability of personal health and clinical assessment and background to the science and statistics are provided in the present inventor's co-pending provisional applications serial No. 60/258,262, filed Dec. 26, 2000, entitled “Method of Administering ChEIs for treating Alzheimer's Disease”; serial No. 60/274,981, filed Mar. 12, 2001, entitled “Method of Drug Development for Selective Use with Individual, Treatment Responsive, Patients;” serial No. 60/301,526, filed Jun. 28, 2001, entitled “Method of Drug Development for Selective Use with Individual, Treatment Responsive, Patients and the Applications of the Method of Drug Development in Medical Care;” serial No. 60/310,058, filed Aug. 3, 2001; entitled “Method of Reliable Measurement in Medical Care and Patient Self Monitoring; international application no PCT/US01/49457, filed Dec. 26, 2001; and “Method for Reliable Measurement in Medical Care and Patient Self Monitoring,” serial No. 60/391,492, filed Jun. 25, 2002 which are hereby expressly incorporated by reference as part of the present disclosure. Other and supplemental methods of analysis could be used as part of this method of reliable monitoring of personal or patient health status and the effects of personal or prescribed health practices, procedures, interventions, treatments. In broad terms, the present invention is directed to a methods of establishing reliable health and disease assessment and using these reliable assessments as grounds for applying health and disease research findings in personal health care, and facilitating the use of these improved methods by electronic or other systematic methods for integrating and processing information in order to encourage the applications of research in self and patient care.

### DETAILED DESCRIPTION OF THE PREFERRED EMBODIMENTS

The present invention is directed to the method of establishing the reliability of any forms of assessment used in health or medical care such that a plan for ongoing assessment takes into account the errors of measurement such that the assessments as used reflect the true condition of the person or patient assessed. In self care or patient care by a physician the selection of appropriate outcome measures takes into account the precision of measurement needed to achieve the health aims with acceptable reliability. This may require that researchers, prior to the public or physician in practice using a method of assessment, use the measure or test at sufficient regular intervals in independent reliability studies or prior to and during health or medical research studies or clinical trials so that a regression line, mean response, or similar scalar summary statistic can be calculated for each individual with adequate reliability to meet the requirements necessary to meet the health care purposes of the assessments. This may also require that the public or physician in practice using a method of assessment, use the measure or test at sufficient regular intervals and calculate the reliabilities of the examinations, tests, scales, or measures in their use to assure their compliance with the reliability requirements developed in a clinical trial or other scientific medical study or to assure that the examinations, tests, scales or measures have sufficient precision to be valid indicators of the individual's true health status or sufficient precision to meet the needs of the analyses or interpretations in which they are used.

The method of invention establishes the practical reliability of health and clinical examinations, tests, scales and other measures by identifying the error component of measurement. This includes in research and clinical patient care a pre-trial, pre-research or pre- or intra-patient care study of different methods of examination, testing or measurement used in different combinations of measures to compare the error components of single measurements and multiple assessments combined in a summary descriptive statistic. The precision of each single or combined summary descriptive statistic is compared to the aims of the research or clinical patient care to select an examination or test and its method of use. In selecting a level of precision of measurement the informativeness of its use, or the reduction of uncertainty in answering the aims of the study or patient care, for an individual or for a group who will be studied or cared for using the measurements, provides one criterion for the required level of reliability in clinical application. For these calculations reliability coefficients, generalizability coefficients or randomization statistics, an information measure, standard error of measurement, confidence interval of measurement can be calculated using the customary procedures known to anyone familiar with the art and available in published technical sources.

This same approach used in pre-clinical trial research or pre-clinical care reliability study can be applied also with adaptation to any set of data when a tester, individual or examiner administers or has administered already a test, examination, scale or measure repeatedly over a period of time to one individual. To extract the data set for reliability study a line is fitted statistically to the longitudinal data using statistical methods of line fitting such as least squares and making appropriate constraints such as holding the time of administration constant in the line fitting and fitting by adjustment of the test or examination scores. The fitted values are then subtracted from the actual values creating a longitudinal data set with zero slope and zero curvature. If further adjustment is needed it is carried out. The residuals are then used as the data set and a mean and standard deviation for the data set calculated. The standard deviation is the standard error of measurement for the data set. From this standard error of measurement the further calculations to develop a confidence interval of measurement can be carried out and applied as described below. By trial and error or other methods the distribution of the confidence intervals of measurement for different standard deviations and numbers of observation can be calculated to guide the user in evaluating the confidence interval of measurement obtained from any one data set.

This same approach used in pre-clinical trial research, pre-clinical care reliability study or the retrospective study of an individual's data can be applied also with adaptation to any set of data when a tester or examiner or testers or examiners administers or has administered already a test, examination, scale or measure repeatedly over a period of time to more than one individual. Two approaches can be used. As already described a standard error of measurement and confidence interval of measurement can be found for each individual and a mean and distribution statistic for the total data set calculated. Also reliability coefficient, generalizability coefficient, or randomization statistics can be used to find a standard error of measurement and confidence interval of measurement.

The method of invention uses the precision of measurement expressed in a confidence interval or measurement or any other statistically appropriate form to monitor a patient's clinical course for stability and for statistically and clinically significant change. The clinician, consistent with the aims of patient care, or the individual consistent with the aims of health care or personal well being, states a criteria of statistical significance that will provide the separation of chance variation due to errors in measurement from improbable deviant scores indicative of change in the patient's actual clinical condition as consistent with the aims of disease management or health care. A confidence interval of measurement or equivalent statistic expressing the criteria of statistical significance defines the measured clinical examination or test variance due to expected error. Thus, if a patient's measured course falls outside the confidence interval of measurement for a course predicted from past experience then the deviation can be assigned a probability and considered as evidence for a statistically significant change. According to the aims of the study or treatment or ongoing monitoring the user sets criteria for clinically significant change or measurements or health significant change or measurement values. These criteria reflect current scientific and medical knowledge of disease management and health. These criteria define the clinical significance of a change. It is acknowledged that the user can judge the statistical and clinical significance of change; the methods of invention develop statistical and scientific evidence to ground these judgments.

Using these methods of monitoring a clinical course a user can detect stability and instability, changes in course, and assign a probability for chance occurrence using statistical probability theory and published technical references. These methods are useful to monitor health for indicators of disease, to monitor disease for changes in the clinical course, to monitor treatment for changes in effect and to detect the effects of interventions or treatments or events on a clinical course. If an individual or physician plans or unplanned experiences an intervention under open or blinded conditions for the introduction of the change these methods can monitor or retrospectively evaluate the effects of the intervention. A course deviation from a predicted course by more than the confidence interval of measurement expressing the interval associated with the criteria of statistical significance meets the criteria of statistical significance for rejecting the hypothesis that no change occurs allowing consideration of an actual change and its interpretation. These methods make the n-of-1 trial more practically useful in patient care as scientifically and statistically sound tool for judging effects from a change in treatment, introduction of a treatment, withdrawal of a treatment, combination of a treatment is available in the deviation of the course from a course and confidence interval of measurement projected from the pre-change experiences of the individual. The individual or physician may use an n-of-1 trial combined with these methods in the course of patient care or health care by employing a third party such as a pharmacist to provide the drug or device and placebo or competing drug or device during a period or periods with the subject, physician, evaluators and others blind to the treatment the individual receives.

The patient course in each of these above conditions and in the conditions described in the incorporated preliminary applications using confidence intervals of measurement can be compared to published patient or average patient courses and distributions from clinical trial, observational and other research to identify potential opportunities to benefit a patient by selecting a drug, treatment, management, dosing, or other intervention indicated by the research as showing potentially more benefit of effectiveness to the patient. In this comparison a confidence interval of measurement matched for the conditions of use of the measures to the conditions in the research can be used to indicate potentially statistically significantly different results in research compared to the individual patient. Criteria of clinical significance can identify significantly different outcomes taking into account a balance of risks and potential benefits.

Each of these methods and steps can be incorporated or embodied in a software or hardware program for a user to interact with. One aim of this invention is to make statistical and scientific resources and methods described herein readily accessible and usable by the professional and general public in clinical and health care. A software program can use the internet or other electronic or mechanical means to query and prompt a user to identify aims, purposes, constraints, interests and to use the methods described to help the user achieve these aims purposes interests within the constraints identified by the user.

As an example persons seeking to reduce their weight to medically recommended levels for their body type can be assessed using the measure weight on a scale once, three times, or more times, on as many successive days as needed to establish the error of measurement in the individual's use of the scale. The individual can then weigh at weekly or other intervals to establish first monthly periodic cycling or variance and then seasonal or yearly variances. Line fitting to the data, then removing the slope by subtracting the data predicted by the fitted line from the raw data leaves residuals. Means and standard deviations can be calculated for these residuals, the standard deviations are standard errors of measurement. With reference to a table of a Gaussian distribution a confidence interval of measurement to meet any criteria of statistical significance can be calculated.

Using these methods or other methods referenced herein the cumulative evidence of error contributions in a data set is used to adjust subsequent measurements for error by providing a confidence interval at an acceptable level of probability (p=0.95 for the confidence interval is customary in medical research and would thus be appropriate here except when local conditions make a different probability appropriate). As a result of the confidence interval of measurement error single or multiple assessments may be required as part of a plan of assessment. The plan requires numbers and times of weights (or for other variables to which the methods in the example may be applied) based on the reliability needed to display the trend of weight over time with sufficient certainty to allow the user to use the trend as an indicator of compliance with a plan for weight reduction, gain, control, according to the user's aims. A device or system is provided that shows over time the longitudinal course and confidence intervals such that error variance can be used to distinguish random or covariate related systematic or periodic changes from statistically significant deviations from the program course to maintain, to increase, to reduce, or once at the sought weight to maintain, weight. The program or device also displays the course in relation to criteria that express the health aims: in the case of weight ranges appropriate to individuals of this individual's body mass and the probable health outcome changes that accompany deviations from the optimal research evidenced weight. Thus the course of the individual can show a prediction of how soon the current program of change will reach each category of outcome, the relation to the planned course, the long-term implications of deviations from the planned course of weight change. The display will show confidence intervals and thus reassure the user of innocuous random deviations but identify the need for attention when weights, improbable as part of the chosen or actual course, indicate significant deviations. The device or system can also be updated with new medical research and display the implications of the new findings for the individual's current and optional alternative courses over time. Similar devices or systems or programs are developed for other outcome measures, commonly employed measures of health and illness, medical tests, examinations, scales whether self administered or administered to the individual being tested, medical or other human activity monitoring instruments, or any other form of health related assessment. The method is described first and then its implementation in specific assessments.

The method of invention in this application uses the methods of pre-trial reliability studies and methods of deriving an assessment plan described in the present inventor's co-pending provisional applications expressly incorporated by reference as part of the present disclosure above. The method of invention in this application can also use methods of retrospective or ongoing iterative analysis of measurement data accrued to the present to establish the reliability of a set of examinations as described herein. The method of invention in this application can also use the methods applied in pre-trial reliability studies and methods of deriving an assessment plan described in the present inventor's co-pending provisional applications expressly incorporated by reference as part of the present disclosure above but applied to a data set of practitioners' or evaluators' clinical ongoing evaluations of patients or subjects to establish the reliability of a measure, examination, test, or scale in the hands of an examiner or examiners in clinical practice. In the current invention each of these methods are applied to the problems of providing reliable assessments of health and medical status and reaching personal and professional medical decisions in response to health aims or evidence without having to depend on unsystematized personal and professional judgments of unknown reliability. The current invention applies and adapts as the situation requires the reliability methods already applied in clinical trials to personal and professional patient care in situations where clinical trial evidence is not being applied to the individual or where the individualized analysis of clinical trial or other medical or health evidence is not available as a model for personal or professional choice of methods of analysis, study, assessment, or decision making.

The method of invention consists of the following basic steps. A device or system or program processor for data or program to structure design and execution of care using the methods described herein is provided for the user the device or system or program enabling the user to proceed through the following steps:

1. Specifying Health or Medical Aims

The user specifies the aims for his or her health or disease management program. These aims lead to

a. the choice of a measure, examination, scale, or test that can be defended as validly reflecting the individual's status in relation to the aims,

b. criteria for achievement of the aims by which the individual can determine success or failure reaching health aims and

c. statistical significance criteria or the level of chance occurrence of a measurement tolerable under the conditions of the application by the individual.

2. Choosing a Method of Assessment

a. All assessment depends on a variable, outcome measure, specific test, scale, examination or other measure that must have validity as a direct measure of a health aim or as a surrogate for the aim. An assessment can be chosen because of its established place in medicine—such as methods of blood pressure measurement, blood glucose measurement, scales of cognitive performance or physical performance—or as an innovation. Reliability studies in groups may be needed to demonstrate adequate reliability and validity for the measure or scale to even be considered as a measure for specific individual health aims. These studies are carried out and then applied in individual care using the methods of pre-trial or ongoing reliability study described in the incorporated applications and herein. Group reliability studies may not be available or the applications of their results may be questioned in which case the provider of health care or disease management will use the methods of invention to establish the precision of the test or/and the precision of the test used by the test administrator with a specific subject, the latter two may be the same person. Thus the method of invention calls for the manufacturer or provider of the system or instrument to demonstrate or to provide the tools for the user to demonstrate the adequate precision of the measure for the purposes it is put to in health care as part of the tools provided to the user. The user then chooses the assessment and the instrument to provide the assessment and the system, device, or instrument needed to support determining the precision of measures and their interpretation by comparisons to research data or other aggregated patient or subject data.

3. Determining a User Specific Error of Measurement and Covariate Effects

a. The measurement instrument or system (hereafter called assessment system) provides a program or procedure for the user to take the measurements on repeated occasions such that the test-retest precision and error of measurement for the user can be calculated. The assessment system can also practice the user with application of the measure until the user's error of measurement falls within the confidence intervals for the group error of measurement obtained following the procedures in 1a or reaches a level required by the informational aims—need for reduced uncertainty—of study identified under 1. The errors of measurement can be calculated using reliability coefficients, generalizability theory, randomization statistics, as appropriate to the measure and application as would be understood by anyone skilled in the arts of statistics and measurement. The errors of measurement also can be calculated for an individual by removing trends in the data set with line fitting and subtracting the time trend. A mean and standard deviation can then be calculated for the residuals. Since the standard deviation represents the standard error of measurement for the data set the reliability can be expressed and confidence interval of measurement calculated as would be understood by anyone skilled in the arts of statistics and measurement. Calculating a standard deviation for the residuals after removing linear trends in the data set is useful for estimating the confidence interval of measurement for a data set from one individual with repeated testing with a measure and for comparisons across individuals. Combinations of data into descriptive summary statistics and iterative calculations of reliability as the data set is enlarged with new measures are useful for establishing the conditions for an assessment plan that match the aims of the evaluations. Generalizability theory is useful because variance from other variables and error variance can be calculated simultaneously. Randomization statistics are useful where exact probabilities are sought with minimum underlying assumptions about the characteristics of the data set and sources. The methods of determining error of measurement are both known to one skilled in the art and described in the incorporated applications and references therein to earlier work.

4. Setting the Assessment Plan

a. An assessment plan is developed and demonstrated by simulation or application to provide satisfactory precision for descriptive summary statistics for the individual's course over time. According to the intended use of the repeated measures different statistics, means, medians, ranges, slopes, curves, may be appropriate as known to anyone familiar with the arts of statistics and measurement. Each data point at a given time may require more than one application of the assessment to reach satisfactory precision that sufficiently narrow confidence intervals of measurement will be found. The assessment plan developed by the assessment system indicates to the user the summary scalar statistic, the frequency of assessment and its summary into data points and the confidence intervals of measurement that will describe the probable limits of range due to error in the course of the individual.

b. Researchers, health care providers, consumers may choose to determine the relative informativeness of different descriptive statistics. Informativeness can be calculated by first calculating the average prior amount of uncertainty over all possible cases in a distribution of possible outcomes for the aim(s) defined for the intended intervention(s). In information theory the average amount of uncertainty is the negative sum over all possible cases of the probability in a distribution times the log of the probability in a distribution for each case. The average posterior amount of uncertainty is over all possible cases in a distribution of possible outcomes for the aim(s) defined for the intended intervention(s) after the information is provided by the measure. The confidence interval of measurement effect is taken into account as a source of uncertainty in calculating a reduction in uncertainty. The confidence interval of measurement affects the certainty of events and thus by increasing posterior uncertainty reduces the information content available. Thus reducing the range of a confidence interval of measurement will in general increase the informativeness by reducing uncertainty. This measure of informativeness as reduced uncertainty in aims can be used to compare the effectiveness of measures with different confidence intervals of measurement and also to compare the effectiveness of research study designs reaching the aims of the users.

5. Criteria for Achievement of Health Aims or for Clinically Significant Health Effects

These criteria judge the adequacy of change in health status or the desirability of a given health status. They may take different forms according to the aims, measurements, health or disease implications of assessments. Existing scientifically evidenced medical knowledge provides the standards for the criteria. The hierarchy of scientific sources for medical knowledge is discussed in evidence-based medicine. (Guyatt et al., 2000)

a. Normative or Idealized Criteria

In some areas of self-care or patient management a population based or research based limit provides a criteria for judging the health status. Examples are blood pressure where specific upper limits are set as healthy. With these criteria the patient is categorically ill or well although subcategories of risk and borderline categories can be defined.

b. Change Criteria

In some areas reduced deterioration in the patient's condition or a trend towards an optimal health status may provide criteria. An example may be weight change in obesity where rate of change or a number of pounds lost each month provides an intermediary criteria until the patient reaches an optimal or acceptable range of weight.

c. Range Criteria

In some areas a range of measurements for the individual may describe desirable optimal health status on a variable and adjacent ranges relative changes in outcome risk. An example is weight where for each body mass medical research can define optimal levels associated with positive health outcomes and for weights above or below increasing risk of negative health outcome.

According to the state of health or disease or the assessment an outcome is provided and updated preferably with new medical research findings. The assessment system displays the user course in relation to the chosen criteria and using the confidence intervals of measurement and clinical expertise on mediating aims and means to the aims displays the user's probable status at present and at future time points if current change or stability persists.

6. Criteria of Statistical Significance

a. Since all scientific evidence receives only probabilistic support the assessment system provides methods for the user, or physician, to set an appropriate level of chance occurrence. This choice defines the confidence intervals used in the estimation of the true course of the user on the variables measured.

These methods of invention present the self-care user or physician supervisor of care with improved opportunities to evaluate whether a health practice is justified by the changes found with instituting the practice for the individual. The study of error of measurement and covariate effects (such as cyclical changes over periods of time) lead to confidence intervals of measurement for the clinical course plotted from the point measurements or statistical summaries of multiple measurements at each point. An intervention followed by a course over time that deviates from the earlier course can be characterized by a probability of occurrence of the post-intervention course as an extension of the pre- or non-intervention course. If one course better approximates the Criteria for Achieving Health Aims or Criteria of Clinical Significance then that course has validity as a desirable health-engendering outcome. The preferred course can be characterized as an improbable random variation from the non-preferred course but the user may wish evidence that the different practices, the intervention conditions, are required to achieve the health aims. Using open random or systematic alternative conditions or blind and randomly sequenced alternative conditions the user can determine the probabilities using the confidence intervals of measurement. An n-of-1 trial is an example. The use of confidence intervals derived from reliability statistics, generalizability theory, randomization tests, and analysis of the reliability of measurement in series applications of a test or examination to an individual allows clinical courses to be compared on the probability of occurrence randomly. Thus the risk or cost of an intervention can be balanced against the probability of losing the effect in reaching judgments about health practices and this choice can be reassessed at any time because the user on an ongoing basis has estimations of the probability of the current clinical or health status occurring under the opposite intervention or non-intervention condition or can readily determine the opposite condition effects.

Pre-application, retrospective, or ongoing use of the assessment measure is carried out in the individual to estimate the error of measurement and confidence intervals of measurement are calculated. This pre-application or retrospective or ongoing study of test-retest reliability is compared with earlier group studies to judge how expertly the measurement is being used or if there are possible other confounding sources of unexpected error. The confidence intervals of measurement are then considered in relation to the demands on measurement made by the aims of the health intervention or modeling. A method of measurement can only be acceptable if the error does not interfere with the estimation of the true status of the patient that is required to interpret the success of the health intervention. In a general example where a specific direction of change will be evaluated a 5% level of statistical significance requires a 90% confidence interval of measurement. If health or disease management course monitoring requires stability of course to be evaluated 95% confidence intervals would be chosen if a 5% chance occurrence is to be distinguished since either extreme of the range may be violated. A point summary statistic that occurs with 5% chance expected occurrence is identified as statistically significantly different from those within the confidence intervals. The Criteria for Optimal Health or Clinical Significance are indicated in a display or taken into account to monitor outcome.

A clinical course is plotted from repeated measures over time and the confidence intervals of the course indicated. The course indicates progress over time towards a Range of Optimal Health or Health Criteria or that the individual remains in the required range. It can occur that for example, before time 3.5 the individual shows a course where the confidence intervals do not overlap the Range of Optimal Health—the individual has less than 5% chance that her current health status complies with current medically acceptable criteria for health. After an intervention at time 3.5 the individual's experience falls within the confidence intervals of a projected plan of correction. By time 4 the confidence interval of the earlier course no longer overlaps the current estimated course for the individual—the individual is assured that there are 19 chances out of 20 that the intervention is having the desired corrective effect on her original health state. At about time 7 the individual reaches the Range of Optimal Health and then adjusts the intervention to maintain this level of measurement. During the correction and after reaching the Range of Optimal Health deviations of single point summary statistics from the overall course fall within the confidence intervals of measurement and the individual is reassured that the overall plan has not been compromised. A measure outside the range of the confidence interval of measurement expected to occur one chance in 20 may be followed by subsequent measures within the confidence intervals of measurement and while in itself improbable it does not evoke a change in the plan of correction.

Specific applications of the method of invention are given as follows with the modifications required by the specific application listed for each. Unless otherwise specified in each area the same basic method is applied for the variables appropriate in the area. The user specifies a health aim based on medical research evidence provided in the device or system or known to her from reports, chooses a method of assessment, determines the specific error of measurement and confirms with the system that the user specific error of measurement falls within the distribution of error of measurement determined in trained users. Based on the aims, a weight change or range to be maintained, the system calculates the number of measurements required at each point and using data from the group test-retest reliability study determines the assessment plan for the user such that the measurements do not interfere with each other or produce interfering carry-over effects. The user adopts from research evidence provided in the system or from external sources criteria for achievement of these health aims or for the clinically significant health effects called for in her aims. The user then adopts from information provided in the device or system or from her physician or personal choice criteria of statistical significance for confidence intervals of measurement and for judging research evidence used to set aims or criteria for aims. The system provides criteria of statistical significance customary in scientific medical practice as known to anyone skilled in the art as default criteria for the system or device. The system then records user measurements over time and for predetermined periods (menstrual cycles entered for each user, seasons, years, and so forth) determines what cycling of measurement occurs as a covariate of time. The system then displays the summary scalar statistic of the course of measurement over time, the confidence interval of measurement for error and for cycling variations, the criteria for achieving health aims, the time points of interventions and any probabilities of courses in relation to each other as described above in examples below:

1. Weight and disorders or diseases of weight

Weight is measured and specific weights targeted in aims and criteria.

2. Blood pressure and disorders or diseases of blood pressure

Blood pressure, systolic and diastolic, are measured and targeted. Individual points, summaries of points or closely spaced sampling over periods of time may be used with a monitor. Thus a curve of blood pressure can be plotted and the area under the curve calculated as an expression of total body exposure to blood pressure. The area under the curve of a normal population can be subtracted to produce a scalar summary statistic of excess exposure to blood pressure.

3. Blood glucose

Blood glucose is measured. A monitor can plot the curve of blood glucose in relation to meals and subtract the area under a normal curve determined in a research study from the user's curve to quantify the excess glucose exposure and, by averaging repeated cycles, specify the times of excess exposure.

4. Cognitive performance

Tests of cognitive performance are used. The user establishes her own baseline by repeated measures and possible cognitive decline can be estimated by comparison of later measurements to the performance at a younger age.

5. Physical performance

Tests of physical performance are used.

6. Mood

Tests of mood, depression, anxiety, tension, agitation are used.

7. Activity or exercise

Monitors of activity or movement or reports are used.

8. Arthritis

Subjective reports, questionnaires, or rating scales of symptoms and disability are used.

9. Stress

Subjective reports, questionnaires, rating scales, measures of skin conductance, temperature, muscle tension, or other indicators of stress are used.

10. Diet

Reports of intake or specific reports of specific targeted dietary components are used.

1 1. Schizophrenia

Rating scales, questionnaires, check lists or other methods of determining the presence and severity of symptoms are used.

12. Diagnostic criteria.

Critieria of diagnosis are used.

13. Management decisions

In the management of any disorder research based or clinical criteria are used.

Examples can be provided of the use of the method of invention in each area:

1. Weight

An eating disorder patient engages in excessive dieting, excessive exercise, purging and other behaviors to limit caloric intake with loss of weight. An assessment system presents the recommended weight range for a person of the user's body mass and the plot of the user's trend of weights. The user is confronted with, and hopefully reassured by, the trend of weight and confidence interval as evidence that she is not gaining excess weight. She also has the evidence presented of probable negative health outcomes from her weight compared with other weights for a person of her body mass. Consistency in management is supported by the evidence of trends of weight and the confidence interval of error compared to individual weights when the user is under professional care.

Professional and family management and self management of these difficult patients can be facilitated by the statistical evidence of changes in the course of weight over time. The individual establishes the confidence interval of measurement. Weight variations around a projected course for maintenance of weight or weight gain are accepted as expected random and systematic errors so long as they fall within the confidence intervals of measurement. One value outside provides evidence towards a deviation from plan that can be confirmed or disconfirmed by subsequent readings. A trend towards deviation that will become statistically apparent can be detected by a program that fits lines to subsets of data thus detecting a trend difference from the long-term data before any one, two, or more measures become statistically deviant.

2. Blood Pressure

A manufacturer develops a wearable monitor for blood pressure. The monitor is used to provide 24 hour curves of blood pressure. Data are gathered on a normotensive population and the area under the curve for this population becomes the target of blood pressure control for a hypertensive population. A hypertensive patient is placed on a medication after a one month baseline of blood pressure recordings. The probability that the post-intervention course occurs as a random error of measurement of the pre-intervention course is plotted with the scalar summary course over time. The target selected by he physician is a user course of blood pressure with the mean of the normotensive population within the confidence interval of measurement of the user's course reached in six months. The drug therapy is managed to reach this outcome.

3. Blood Glucose

With a monitor available that continuously samples for a patient's blood glucose a drug company uses the method of this invention to record the daily blood glucose profiles of each research patient. By subtracting the area under the curve of a normal range of blood glucose from the area under the curve of each patient's blood glucose profile the research provides both individual profiles of response and the incremental accrual of, and total daily, excess glucose exposure over time within those profiles. The research goes on to key these exposures to surrogate markers of complications by long-term follow-up of research patients and from other research sources. The data from a monitor worn by a patient is entered into his electronic medical record over the Internet and interpreted with the method of this invention. The doctor. then can evaluate the patient against a research or practice derived data base and achieve closer control of blood glucose with medication or interventions. The extent of the problem of inadequate control of blood glucose is available each day and does not have to wait for tests for glycosolated hemoglobin. (Bakerman, 1984, p. 226) A patient, by viewing the progression of daily blood glucose plots over months of treatment becomes reinforced in his adherence to the management regimen by the evidence of progress and the immediate increased probabilities for worsened outcome when poor glucose control occurs.

4. Cognitive Performance

An aging person wishes not to have a subtle progressive cognitive defect interfere with management of business finances, personal finances and decisions, preparation of income tax. The person determines with the reports of others and a physician's examination that he has no immediate deficits. He uses a measuring scale of cognitive performance in an assessment device or program repeated to establish an error of measurement and any cycling effects. He then programs the monitor to query him using the measures over the future such that it could detect a statistically significant difference in any three month period. With this programmed assessment the user is reassured that deviations that could result in otherwise undetected disability will be brought to his attention or to the attention of others.

5. Physical Performance

A patient with a potentially progressive neuromuscular condition must maintain flexibility with regular stretching exercises. Using exercise equipment that monitors joint range of motion a baseline is established and then any trends that potentially may significantly deviate are identified. The user specifies a trend that would reach a 5% deviation from the confidence interval of measurement within 4 months should be brought to her attention. The user can pursue her daily routine with confidence that any indications that it will not be adequate to her health goals will be brought to her attention.

6. Mood

A physician diagnoses a patient as depressed but is uncertain, as is the patient, if the depression is due to circumstances in the patient's life or presumed genetic-biochemically mediated factors that operate independently of her current situation. They agree to medication and to monitoring with a self-rating scale and a physician rating scale. The scale measures are administered in compliance with an assessment plan developed after determining the confidence intervals of measurement and the results imputed to a monitor. The patient and physician enter life stresses into the monitor. The plot of measurement is studied for stability of a progressive change after drug intervention and presence of statistically significant changes after stresses using the methods of invention. The probabilities of deviation of the clinical courses provide evidence that statistically significant change is more probable in relation to stress than to drug. This directs the physician towards looking to life stresses as the source of the depression.

7. Activity or Exercise

An individual reads in a popular report that expending 15% of caloric intake in exercise at 50% of maximal heart rate improves longevity by 10 years on average. The individual adopts this as a health aim. She determines maximal heart rate on an exercise machine and chooses to use the machine for daily exercise. She estimates caloric intake and the machine provides her a target range of activity. Each day she exercises to near the target range and uses a monthly average to expend the target calories in exercise. She monitors her compliance with the criteria that the 95% confidence intervals of measurement for the course of exercise monthly should overlap the target range she has set from the medical research findings.

8. Arthritis

A patient on anti-inflammatory medication finds difficulty adjusting medication based on unaided judgment because she thinks reactions to personal problems interfere with her independent assessment of the severity of arthritis. She chooses to use a self-rating symptom scale and a physician's recommendation for use of medication in relation to scale measurements. With the confidence intervals of the plotted reliable measures she can better stabilize the dosing of medication over time and improves control over arthritis symptoms.

9. Stress

A person feels that stress causes him personal distress that could be relieved if he could reduce his sense of stress. He adopts a measure of stress and an intervention to relieve stress. He uses an assessment system and then gauges the amount of intervention by the progress towards aims for reduced stress he has set. He finds the assessment system and the methods of invention helpful because the presentation of incremental change progressively provides the reinforcement he needs to persist in the intervention and overcomes his personal tendency to reach his goals with unrealistic haste.

10. Diet

Health evidence suggests that some types of cancer are less common among Japanese who live in Japan and follow the traditional Japanese diet than among Japanese who live in the United States. Still higher risks are reported for non-Japanese Americans. Population studies support diet as important to cancer risk. An individual decides on the basis of the evidence to introduce into her diet soy in the forms used in Japan and in the amount where she will use soy at the same percentage of total calories used in Japan. To implement this health aim require a number of estimations on the users part. She estimates calories based on her weight and activity measured by monitors she has available. She uses a health monitor system into which she enters the quantity of soy and type eaten each day. The system calculates the soy calories and estimates total calories from weight and activity providing the percentage of soy. She selects the percentage target from the percentages reported in Japan residents, Japanese Americans and non-Japanese Americans such that the user's diet contains the mean amount ingested by Japan residents but above an amount ingested by 66% of Japanese-Americans based on recommendations in the epidemiological studies. The relevant evidence from these studies is updated into her health monitor as described herein. Her assessment system provides her a longitudinal report of her caloric soy intake and deviations over whatever periods she selects them to be averaged. This provides an example that can be generalized to any dietary components as a means for monitoring intake.

11. Schizophrenia

A patient in a supportive employment and case management program fears that his gains will be lost to his symptoms. He wishes to contact staff at first signs of relapse but knows the literature that evidences support that one of the problems with relapse is increasing isolation from support. He therefore uses a self-rating scale. The use of the method of invention provides an objective measure for when he should call for help—a trend the would reach statistically significant deterioration within 5 days since his past experience shows he decompensates in 5 days. Filling out the scale every morning and evening a trend can be detected within 24 hours. As a backup he arranges for the results to be sent electronically automatically to his case worker. When he shows a trend towards deterioration his caseworker appears that day at his supported employment site and they can successfully avert further progression.

12. Diagnostic Criteria

One element in diagnosis of infectious hepatitis is the presence of evidence supporting virus such as antibodies to virus or demonstration of virus. Other elements are elevations in serum concentrations of hepatic enzymes and hepatic excretory and synthetic products. Each of these contributes to the probability of disease. A patient suspected of disease can be monitored and the course of enzymes, viral load can be monitored using the confidence intervals of measurement. Interventions can be probes to determine whether a static or dynamic state underlies the monitored measure. The method of invention allows a changed trend to be given a probability of occurrence under pre-existing conditions and the trend of measurement. The error of measurement and cycling variation from covariates is removed by the method of invention providing a reliable measure of the true course of the patient. This allows a specific clinical course to be associated with specific risks for outcome improving on the probabilities of active disease derived from group studies and the more generalized predictions available from these group studies. (Woodley and Whelan, 1992, pp. 309ff.)

13. Management Decisions

In myocardial infarction where serum enzymes are used to suggest, reach, confirm, exclude, or follow the course of suspected infarction even the best indicators are only relatively specific and the clinician's patient is compared to summary group statisitics from research studies. The research studies qualify a test as a reliable and valid indicator of disease process being present in a group affected by the condition compared to a control group. The clinician calls on the test as part of the clinical evaluation of the individual patient in his or her care and uses clinical judgment to assess the implictions of the research validated test for the individual patient. (Woodley and Whelan, 1992, pp. 87ff.) In therapy of myocardial infarction research predicts the probability of restoring blood flow as a percentage of all patients treated, or markers of therapeutic activity of multiples of initial measurements in a patient based on improved group outcomes associated with similar directions or magnitudes of change in research. (Woodley and Whelan, 1992, pp. 91 ff.) Even the identification of high-risk patients is based on group comparisons not a model for individualizing prognosis. Group stratification is used. An example is provided by the Killip classification of myocardial infarction. (Woodley and Whelan, 1992, pp. 88) Rather than categorical classification using the method of invention the physician can follow the trends of measurement for each of the variables, or combinations, and use early detection of changes in trend with the probabilities provided by the method of invention. This example illustrates how the method of invention elevates the level of measurement, in this case from categorical to orders and possibly intervals or ratios in some applications.

Examples can also be provided how health aims may require application of the methods of invention to many different areas simultaneously. For example, a favorable profile of blood glucose induced by drug may prove to have different long-term outcomes predicted for a patient who diets, exercises and loses weight while the same initial profile of response will deteriorate and have an increased risk of secondary consequences of diabetes mellitus in a patient who does not observe dietary restrictions, exercise, and lose weight. Or in two patients who differ only in not losing weight even though they diet and exercise, the same degree of initial research control of blood glucose may have different long-term consequences in followup because initially one patient was 5% below optimal body weight and the other patient was 40% above optimal body weight. Thus the interpretation of one measure may depend in different areas of health or disease on interactions with other measures determined of importance to long-term outcome in research studies. The assessment system is in all cases reflective of the current state of medical knowledge and would provide this multivariate informed interpretation to the user.

In the method of invention an item of data for an individual may be the following:

1) A health or medical assessment at an instant in time, for examples, a blood pressure, a laboratory test result, a score or single response to a question or other stimulus, or any other result from a medical examination;

2) An aggregated score or response where established methods provide a questionnaire or rating scale score, a summary score or quantification of a laboratory or imaging or other medical study of the patient;

3) A profile of the patient over time, for example a defined time period, of an hour, day, week, or other period of time where an aggregated measure(s) over the time become the unit of repeated measurements, comparison, and analysis; or

4) Any other information used to assess treatment efficacy or health status in medical practice or research.

A general example of the application in clinical and health self-care practice is provided in the following illustration. We assume the person is under the care of a physician who has, with the patient, set aims for the patient's progress as indicated by self-monitoring.

Dr. Jack instructs her patient Mr. Reed to self-monitor his blood glucose and blood pressure regularly as part of the management of Mr. Reed's Diabetes Mellitus Type II and Essential Hypertension. Dr. Jack recommends to Mr. Reed dietary restrictions, an exercise program, goals for weight loss, and prescribes an oral hypoglycemic medication and an antihypertensive medication. Mr. Reed uses monitors for blood glucose and blood pressure designed for the home. These monitors are integrated with a Personal Health Profile Monitoring and Assessment system the method of invention described herein. The assessment system after establishing an assessment plan based on study of the errors of measurement 1, integrates the reports of Mr. Reed's self monitoring into the system and analyses the findings in relation to criteria of clinical significance of measures; 2, communicates the findings to Mr. Reed's electronic medical record in Dr. Jack's office; 3, indicates to Mr. Reed and Dr. Jack when statistically significant deviations from acceptable clinical practice occurs; 4; receives and integrates into its assessment new medical evidence relevant to evaluating blood pressure or blood glucose control; 5,provides data and analyses helpful in evaluating weight, activity measures, dietary data, in relation to the aims set in the original plan of assessment.

Mr. Reed uses a weight scale without electronic programming so he enters his weight into a web site that provides a program to analyze his data for reliability and to predict his future health outcomes from his clinical course. The web site program plots the weight goal chosen by Mr. Reed and Dr. Jack and shows his course in relation to the goal Mr. Reed selects. As his data accrues over time the web site program calculates a confidence interval of measurement. Since Mr. Reed had historical data the web site indicates his course and the time of his health interventions. A line-fitted to the data indicate a 5 pound annual mean weight gain historically and projected. His new data trend below this line but fail initially to show statistical significance because of a large confidence interval of measurement. Regardless the program calculates a better fit than the original projection for the last four weights projecting a statistically significant difference by six months. This encourages Mr. Reed that his efforts show benefit and that his program will achieve the desired change over the two years his physician gave him to reach his ideal body weight.

Because the confidence intervals in the data processing within software and hardware programmed monitors and the web site programming available to doctor and patient and the public indicate to Mr. Reed and Dr. Jack when statistically significant deviations from a projected clinical course, when violations occur of a constraint on variations that either wants to avoid, when deviations from acceptable clinical practice programmed into the system occur and values not significantly different but only varying within expected error, either can carry out an n-of-1 trial. Dr. Jack decides to evaluate the effectiveness on blood glucose of a supplemental medication when Mr. Reed complains of uncomfortable adverse events accompanying the introduction of the medication. Dr. Jack uses a program in the Personal Health Profiles web site that assigns periods with and without a supplemental medicine or other intervention in conformity with constraints entered but with the doctor and patient blind to the exact dates. The program automatically notifies the pharmacy to prepare matched drug and placebo and when to dispense each. Dr. Jack and Mr. Reed monitor his blood glucose and then after the trial period the web site program analyzes the daily area under the curve of blood glucose, or any other outcome parameter chosen, and plots the data with the projected historical course and confidence intervals. The plot reveals that during the period of blind supplemental drug administration the area under the curve of blood glucose plots fell below the confidence interval of measurement surrounding the projected blood glucose course from past values while during the other periods without the supplemental medication the values were within the confidence intervals. Both conclude that the medication offers additional protection from hyperglycemia but that in view of the discomfort from adverse events and the ease of the n-of-1 trial they will use an n-of-1 trial to reevaluate the need for medication annually since with increased exercise and reduced weight Mr. Reed may bring blood glucose under better control.

Dr. Jack notes the difficulty Mr. Reed has demonstrating a significant weight change and uses the web site program to determine what error of measurement reduction, or precision of measurement would be needed to provide statistically reliable evidence of weight change before two months. He uses the measure of informativeness to determine from the reduced uncertainty needed the required precision of measurement or size of confidence interval. He also notes that this same calculation can be used for an extended clinical trial he is planning since the patients within one confidence interval of measurement surrounding the criteria of clinical significance cannot be categorized as responders or non-responders. He decides he will want to be uncertain about only 5% of patients at maximum and notes he will need a confidence interval for the criteria of clinical significance that will cover no more than 5% of the patient drug treated sample. He then returns to thinking about the scale to recommend and he finds the required precision and finds a scale that he can recommend to patients who wish earlier support of their life style changes.

The method of invention can be embodied in a computer software program or hardware system or any device capable of carrying out the required operations or accessible electronically by the Internet or from another centralized source any of these independent of any specific system or method of assessment, or capable of being interactive with devices or systems of assessment, or integrated in a device or method for assessment, or provided as a published set of directions, flow charts, worksheets, instructions, guidelines, technical training, skill training, or other forms and result in publications in articles and books, audiotapes, CD recordings, or other forms of presentation of the methods of invention.

Software or hardware programming to apply the methods of invention in a clinical trial and to apply the results of the clinical trial in patient care includes as needed procedures to accomplish the following steps:

1) identifying the aims of a clinical trial (CT) or patient care or health care. Each of the following references to patient includes a person in self care who pursues well being or health with systematic interventions in health habits or life style. The aims anticipate applications of the CT, or analysis of patient care, in patient care;

2) identifying proposed outcome measures of each patient's medical condition, and determining whether the proposed outcome measures have sufficient reliability to meet the aims of the CT or patient care and the anticipated applications of the CT or analyses of patient care in patient care;

3) conducting a reliability study of at least one outcome measure to be used in the CT or to be used or already used in patient care and determining the error of measurement of the at least one outcome measure based thereon;

4) developing an assessment plan for the CT and or patient care by selecting the frequency and form of measurement of each patient's medical condition based on an error of measurement offering sufficient reliability to meet the aims of the CT or patient care;

5) identifying criteria of clinical significance for use in the CT and in applications of the CT in patient care;

6) selecting criteria of statistical significance to set the level of chance occurrence for use in interpreting comparisons in the CT or patient care;

7) assessing a plurality of patients in the CT or one or a plurality of patients in patient care in accordance with the assessment plan; and further comprising at least one of the following steps:

(i) comparing each patient's clinical course to the criteria of clinical significance, and determining whether the patient's condition is improving or not based thereon;

(ii) estimating the probability that the drug or other medical procedure or health intervention is necessary for improvement of an individual patient's condition by comparing the chance occurrence of each individual patient's clinical course among active and placebo treated patients in a CT or with the use of an n-of-1 trial;

(iii) determining based on at least one long-term outcome of a CT or other observational or other research studies whether the measured improvement will result in a long-term favorable outcome for the individual patient; and

(iv) identifying at least one optimal expected long term outcome, comparing a patient's expected long term outcome to the optimal expected long term outcome, and assessing the probability of whether the patient will achieve the optimal expected long term outcome.

In these steps a study uses test-retest precision with individual patient data and on data from groups of patients as appropriate to the aims of conducting the test. Determining the error of measurement includes determining the error of measurement of a single administration of an outcome measure and the error of measurement for multiple administrations of an outcome measure summarized as a descriptive summary statistic and the ability to compare the informativeness of different statistics in terms of the aims of patient care.

Each patient's clinical course is characterized by the outcome measures carried out in compliance with the assessment plan. Comparing each patient's clinical course to the criteria of clinical significance includes determining whether each patient meets the criteria of clinical significance and identifying each patient as a responder or not based thereon. The steps of assessing an individual patient's response to a drug or other medical procedure used to treat a condition of the patient are: evaluating the patient in accordance with the assessment plan of the CT or patient care; and further comprising at least one of; confirming that the error of measurement for the at least one outcome measure applied to the individual patient does not exceed the error of measurement for the corresponding outcome measure used in the CT or determined from earlier data from the patient or a group of patients; comparing the patient's clinical course to the criteria of clinical significance from the CT or patient care, and determining whether the patient's condition is improving or not based thereon; applying the criteria of statistical significance from the CT or patient care to estimate the probability that a patient is or will become with continued treatment a responder or not based on the criteria of clinical significance; applying the criteria of statistical significance from the CT or patient care to estimate the probability that the drug or other medical procedure is necessary for improvement of the individual patient's condition; determining based on at least one long-term outcome of the CT whether the measured improvement will result in a long-term favorable outcome for the individual patient; and) identifying at least one optimal expected long term outcome, comparing a patient's expected long term outcome to the optimal expected long term outcome, and assessing the probability of whether the patient will achieve the optimal expected long term outcome.

The assessment plan from the CT or a reliability study of patient care data includes information concerning at least one of: (i) whether different outcome measures reliably support the aims of the CT or patient care; (ii) how outcome measures are combined into descriptive summarizing statistics to meet the aims of the CT or patient care; (iii) how frequently outcome measures or combinations of outcome measure administrations needed to form descriptive summarizing statistics are administered to patients; (iv) how multiple administrations avoid carryover effects; (v) which single measure or descriptive summarizing statistic for multiple administrations is used in data analysis to control error of measurement in a test of hypotheses in the CT or in patient care; and (vi) which single measure or descriptive summarizing statistic for multiple administrations is used in describing the individual clinical course of each patient in the clinical trial or in patient care..

A single measure or a scalar summary statistic summarizes multiple measures taken in relation to each other within a predetermined period of time to form a descriptive summarizing statistic. The selected measure or scalar summary statistic describes the patient's clinical course as a clinically significant response or non-response to the treatment received. A confidence interval of measurement is calculated from the error of measurement and criteria of statistical significance and used to judge the patient's clinical course in relation to criteria of clinical significance. The probability that the drug or other medical procedure is necessary for improvement of the patient's condition includes at least one of the following comparisons: (i) the probability that the treated patient's course would occur under both active treatment and comparison or placebo conditions; (ii) whether a confidence interval of the treated patient's course overlaps or does not overlap a mean of courses within an actively treated or placebo treated group in a CT, (iii) an odds ratio of the cumulative frequency of the treated patient's course among actively treated patients divided by the cumulative frequency among comparison or placebo treated patients in a CT; (iv) an exact probability comparing the treated patient to active and placebo treatment determined by a randomization test; and (v) another comparison required by at least one aim of the CT, patient care, or intended use of the treatment in patient care. Estimating the probability that the drug or other medical o health procedure is necessary for improvement of the patient's or person's condition includes calculating at least one odds ratio for each of a plurality of clinical courses occurring under treatment and placebo conditions in CT data comparisons or for n-of-1 trials with an individual patient. The odds ratio includes the probability that a surrogate outcome indicates a treatment effect will result in a long-term health benefit.

Criteria of statistical significance perform at least one of (i) determining whether an individual patient is a responder or not; (ii) establishing the probability that an individual patient's clinical course could occur under placebo or under active treatment conditions; (iii) statistically supporting the internal validity of the CT, n-of-1 trial or patient care; (iv) selecting confidence intervals; [and] (v) distinguishing as different two or more clinical courses; and (vi) estimating whether a clinical course projected into the future will indicate the patient is a responder or not, is benefiting from active treatment or not, or will have favorable long-term health outcomes or not.

Determining whether an individual patient's condition is improving or not includes at least one of: (i) using n-of-1 trials to confirm whether the patient is meeting criteria of clinical or statistical significance, (ii) using n-of-1 trials to confirm whether the patient is experiencing a clinically significant or statistically significant effect of treatment compared with placebo, and (iii) using n-of-1 trials to confirm whether under an alternative treatment condition the clinical course falls outside the confidence intervals of measurement for a course projected from an earlier or later comparison treatment condition. Confidence intervals for measurement of outcomes from treatment, test for treatment and placebo effects in n-of-1 trials.

Determining whether the measured improvement will result in a long-term favorable outcome for the patient includes generating probabilities for long-term outcomes specific to distinct clinical responses. The distinct clinical responses include individual courses, course intervals bounded by confidence intervals of measurement, and comparisons of an individual to others with courses that fall within the confidence interval of measurement of the individual's course. Differences among courses are measured by surrogate outcome variables with confidence intervals of measurement derived from the error of measurement. Confidence intervals for measurement of outcomes can also be derived from treatment or monitoring experience with a person or patient, and a model for a practicing physician to use to assess each patient's clinical course in relation to established clinical and statistical criteria of significance and individual patient courses in the CT.

The programming system also allows a step of conducting a reliability study includes conducting reliability studies of combinations of outcome measures to determine which number and frequency of administrations of the outcome measures is required to achieve the aims of the CT or patient care. Conducting a reliability study includes conducting reliability studies of alternative outcome measures and combinations of number and frequency of administrations to select the outcome measure or measures for the CT or patient or health self care. Comparing each patient's clinical course to the criteria of clinical significance further includes assessing degrees of response in relation to the criteria of clinical significance and the probability of a patient becoming a responder or not if the patient maintains the present clinical course into the future. Evaluating the patient in accordance with the assessment plan of the CT or patient care includes interpreting the results of the evaluation in accordance with the assessment plan and patient data generated in the CT or in the course of patient care or health care. It may be preferable to confirm that the error of measurement for the at least one outcome measure applied to the individual patient does not exceed the error of measurement for the corresponding outcome measure used in the CT or historically in patient care or health monitoring. The system supports confirming whether the error of measurement for the at least one outcome measure applied to the individual patient exceeds the error of measurement for the corresponding outcome measure used in the CT, patient care, health care, or otherwise and if so, determines a confidence interval of measurement for that patient.

These steps are implemented by a system with the following major components or routines and subroutines. An educational or informative module to provide instruction in the system and describe the scientific, medical, statistical, and practical grounding for the system; a demonstration module that illustrated the system's features for the user; a user module that allows the user to access and use the resources of the system. The user registers and establishes an electronic record of data he or others submit and or accesses his or her electronic medical record for the data to be analyzed or monitored by the system. The system provides services to diverse populations: physicians and other health care professionals; patients; families; caretakers; researchers; medical insurers; government agencies; disease managers; pharmaceutical manufacturers; the healthy individual. The resources of the system are specially modified to address the different needs of each of these populations of users.

The system provides two major monitoring and analytic resources: disease or health monitoring; and disease or health management. Monitoring graphs health or clinical indicators over time and uses the subroutines of the system to characterize and analyze the courses plotted. Management similarly plots individual data over time but characterizes and analyzes the data using research data from scientific and medical studies. In both of these activities criteria of clinical significance or health significance, criteria of statistical significance, confidence intervals of measurement can be displayed and data analyzed in relation to these. The confidence intervals of measurement are analyzed from pre-clinical trial, clinical trial, or patient care data and use in conjunction with different descriptive summary statistics and informativeness analysis subroutines to display options to the user. Subroutines also calculate odds ratios, probabilities, distribution frequencies as needed to characterize a clinical course or outcome implications.

The method of invention can also provide a Disease Management System and the System can be available as a web site or in any other media that allows the required access and analysis and interpretation for a user.

The Disease Management System provides a range of Management Planning Options to the user. The user selects, according to clinical need, among Clinical Treatment Modules and Assessment Plan elements. Four Management Sequences comprise the Clinical Treatment Modules:

(i) Management Sequence I for Initial Treatment or Intervention and Evaluation of the Effectiveness;

(ii) Management Sequence II for Ongoing Management and Evaluation of the Responding Patient with dispositions available to deal with deterioration in a previously acceptable level of response to intervention;

(iii) Management Sequence III for Management of Deterioration in the Previously Responding Patient; and

(iv) Management Sequence IV for Management of the Non-responding Patient or the patient who does not respond to any treatment approved by pharmaceutical regulators such as the Food and Drug Administration for use in the patient's condition.

To provide care within these Disease Management Sequences the user must develop or adopt an Assessment Plan. Broadly the Assessment Plan uses four groups of Evaluations:

(v) a Pre-treatment Evaluation to establish the state of health or illness of the patient or person prior to a planned intervention or treatment;

(vi) a Post-treatment Evaluation to establish the state of health or illness of the person after receiving the intervention or treatment;

(vii) a Continued Treatment Evaluation to monitor the success of intervention after an initial evaluation indicates the appropriateness of continuing the treatment; and

(viii) Blind and Unblinded N-of-1 Trials to compare treatment conditions when confirmation or evidence of the patient's condition is required to plan further treatment.

These are only examples of Assessment Plan evaluations for the Disease Management System described. Other Management Sequences are used according to the needs of the clinical or health situation. For example, A Health and Clinical course Management Sequence is also available. In this Sequence the user monitors an outcome variable to determine whether specific health or intervention aims are being met. Resources from the Disease Management System can be used—for example an N-of-1 trial to determine whether a change in health practices better achieves health aims such as weight control, strength, balance, blood cholesterol reduction, and so forth.

For any Management Sequence an assessment plan must be developed and validated. Three options for determining the error component-portion of the score or measurement from an examination, test, scale, instrument, or other method of measurement, due to random error—are presented to the user. These methods are obtaining a confidence interval of measurement from a research study or prior to a clinical trial, from a study of a number of different patients in a practice setting, or by repeated retesting of an individual. These options are shown in FIG. I as they are presented as a resource to or an integral part of a Management Plan. The user selects one option as the source for the analysis needed to select one or more outcome measures, the frequency of administration of each measure and the summary statistic for each measure that assures adequately small error components in a score or examination result such that the score or examination result can be a true indicator of the person's actual condition.

To use the resources in FIG. I the user first, for the disease of interest, identifies in predetermined criteria of clinical or health significance that define the health—promoting, therapeutic or rehabilitative—aims of the application of treatment or intervention to be applied in patient or personal care at least one predetermined magnitude of change or lack thereof in at least one outcome measure. This is preliminary to selecting at least one outcome measure that offers adequately precise measurements for the outcome measure to be used as the best available indicator of whether an individual person's or patient's response to a drug or intervention meets the aims of treatment. With these aims the user can use the choices in FIG. I to reach an Assessment Plan.

A user defines an error component of each prospective outcome measure by one of the FIG. I routes to calculating a standard error of measurement:

(i) Research Study (Reference Number 10) estimates error in the outcome measure by performing a test-retest of the outcome measure on a group of research subjects and generates test-retest data on the outcome measure. A reliability statistic and standard deviation (SD) from the test-retest data are calculated and used to calculating the standard error of measurement. If a reliability coefficient (r) is calculated the formula known to anyone familiar with the statistical arts provides the estimate of the standard error of measurement (SEM)—(SEM=SD × square root of (1−r²)). Reliability statistics include the reliability coefficient, generalizability coefficient, or a randomization statistic.

(ii) Practice Group Study (Reference Number 12) uses the same methods as the Research Study but studies a group of persons in a non-research setting.

(iii) Practice Single Subject Study (Reference Number 14) estimates the error in the outcome measure by performing a test-retest of the outcome measure on a single subject and generates test-retest data on the outcome measure, allowing calculation of a standard deviation for the data set. Since there will probably be a small number of score the standard deviation can appropriately be adjusted for sample size using multiplication of the standard deviation squared by the factor developed by dividing the number of observations by one less than the number of observations and then the square root to obtain the corrected estimator or other adjustments known to someone skilled in the statistical arts. The standard error of measurement is the standard deviation or the standard deviation adjusted for sample size.

When the test-retest data taken over a period of time demonstrate a trend in the data points away from the initial estimator of the mean the trend can be removed by fitting a regression line to the data set and subtracting the values predicted by the regression line from the test-retest data to remove the effects of the trends over time on the test-reset data. The above methods can then be applied to the adjusted data set to estimate the random error in the original data set.

To obtain a confidence interval of measurement for a given criteria of statistical significance the user consults a statistical text for, or the web site supplies, the appropriate multiplier expressing the cumulative probabilities in a distribution that correspond to the selected criteria of statistical significance. The error component, expressed as a confidence interval of measurement (CIm), is obtained by multiplying the standard error of measurement by the multiplier to thereby ensure that any measurement with an outcome measure when the measurement falls outside of the error component will occur by chance with an average frequency not greater than the chance frequency defined with the criteria of statistical significance. Other statistics can be used to express the error component or confidence interval of measurement, for example a maximum error in relation to a mean or a median in a data set.

The development of the error component allows the user to call, for purposes of the treatment or intervention addressed with the methods of invention, the measurements that fall outside of the error component by chance with an average frequency not greater than the chance frequency defined with the criteria of statistical significance the true indicator components of the measurement. To develop the Assessment Plan the user must define the best available indicator or indicators of whether an individual person's or patient's response to a drug or intervention meets the aim of a health practice, intervention, or treatment by comparing different outcome measures, different frequencies of administration of different outcome measures, and different summary statistics of different outcome measures to select at least one outcome measure, frequency of administration and summary statistic based on their adequacy to achieve the health aims. The user can consider at least one of the following according to the situation and health aims: the smallest error component available; the smallest ratio of error component to true indicator component; an error component that is less than the change or lack of change from the patient's health state addressed by the treatment to the health state required by the criteria of health or clinical significance; the smallest ratio available of error component to the change or lack of change from the patient's health state addressed by the treatment to the health state required by the criteria of health or clinical significance; the smallest ratio available of error component to true indicator component to the change or lack of change from the patient's health state addressed by the treatment to the health state required by the criteria of health or clinical significance; the smallest ratio of the density of outcomes, whether predicted or known, within one error component of the criteria of statistical significance compared to the density of outcomes, whether predicted or known, outside of one error component at the criteria of statistical significance. The relative densities of outcomes within the error component range surrounding the criteria of clinical or health significance to those outside the range is important since cases within the range will be undecidable with the confidence required by the criteria of statistical significance..

An assessment plan uses the one or more selected outcome measures, frequency of administration and summary statistic for the outcome measure(s) in the manner that makes the best available outcome measure, frequency of administration, and summary statistic the best available adequately precise indicator of the person's actual health status for the purposes of the above aims of intervention or treatment.

FIG. II illustrates the general flow of management decisions according to the results of treatment and assessment in each Disease Management Sequence. Diagnosis of the Patient's Condition (Box 16) leads to Selection of a Treatment (Box 18). Then proceeding to Box 20, Management Sequence I, the user determines the success or lack from treatment. If successful then treatment under Management Sequence I (Box 20) leads to Management Sequence II (Box 22). The person or patient remains under Management Sequence II so long as the success of treatment is maintained. If the initial treatment effects are lost the user proceeds to Management Sequence III (Box 24) to evaluate the appropriateness of continued treatment in spite of loss of initial effects, possible reevaluation in Management Sequence I for a new alternative treatment, or selection of an alternative treatment and its evaluation in an N-of 1-trial or proceeding to Management Sequence IV (Box 26).

If treatment initially under Management Sequence I (Box 20) is not successful enough to proceed to Management Sequence II then an alternative treatment can be selected and tested under the conditions of Management Sequence I or after repeated failures when no approved treatments or interventions remain the user proceeds to Management Sequence IV (Box 26). The decision processes and analyses with each of the four Management Sequences in FIG. II (Boxes 20 through 26) are illustrated in FIG. III through VI. In FIG. III Management Sequence I Post-treatment evaluation (Box 32) leads to continued treatment and Management Sequence II (Box 34) or for non-responders (Box 36) consideration of alternatives (Boxes 38 and 44). If no alternatives are successful in AD after three tries (Box 42) the patient is tested to determine whether any effects occur using Management Sequence III (Box 48). As in all Figures in the absence of any regulatory approved alternatives (Box 44) the user goes to Management Sequence IV (Box 46).

In Management Sequence II (FIG. IV) continued success leads to continued treatment and evaluations (Boxes 50, 52) and failure or possible better response to Alternatives (Boxes 54, 56, 58).

In Management Sequence III (FIG. V) patients are evaluated with an N-of-1 trial (Boxes 60 and 62) and based on results Alternatives (Boxes 64, 66), or Management Sequence II (Box 70), chosen if treatment is better than placebo in the trial, or if not then Alternatives (Boxes 74, 76) or Management Sequence IV (Box 72) chosen. Altenatives are chosen when available, research shows outcomes better than current patient outcomes. Management Sequence II is chosen to continue current treatment. Management Sequence IV is chosen when no other regulatory approved treatment alternatives exist.

In FIG. VI Management Sequence IV consider (Boxes 80, 86) both non-approved remedies (Box 82) and investigational studies (Box 88). Other Management Sequences (Boxes 84, 90) are used as appropriate according to the results of the choice.

The flow of analysis and interpretations for a Health or Clinical Course Monitoring Management are shown in FIG. VII. Aims (Box 92) lead to assessment planning (Box 94) and the desired course (Box 96) for reaching the aim. The course is monitored (Box 98), interpreted (Box 100, 102, 104), and research resources used as needed to confirm interpretations (Box 106)

These Disease Management resources can be exemplified for Alzheimer's disease (AD). To implement the management tools in AD a number of subroutines are constructed as follows:

Subroutines:

1. Data files to hold data

1 a User files for data from users

1a1 Patient files to hold patient data

1b Reference files to hold information needed for references when offering interpretations of analyses of patient data

1b1 Clinically important response overlay-a reference data set for interpretation whether a patient shows a clinically important response-is a responder.

In this reference overlay the program plots a series of lines on a graph (see Subroutine 7. These lines are-(In this example the MMSE refers to the MiniMental State Examination that is commonly used to evaluate Alzheimer's disease patients and uses a mean of three MMSE examinations to establish the error component of the patient outcome measures. The criteria of clinical significance is 50% reduction in MMSE loss and the criteria of statistical significance p=0.05 chance occurrence.)

Line 1 labeled “Estimated Untreated Alzheimer MMSE Loss” This line starts at t=0, MMSE=0 and goes to 6 months with MMSE=−2.0 and at 12 months −4.0 and so forth. Extend line over remainder of graph x axis dimension

Line 2 labeled ‘Criteria of Clinical Significance’ this line starts at t=0, MMSE=0 and goes at 6 months with MMSE=−1.0, at 12 months −2 and so forth. . Extend line over remainder of graph x axis dimension

Lines 3 through 6—CIm s from file 1b2 for first and second lines. Show CIm lines at +/− CIm in relation to line 1 and 2 and associate with shading. May be best as shaded colored areas that allow overlaps to be distinguished.

Area above the Line 2 is labeled on the graph with “Clinically Important Effect-Responder” and area below Line 2 “No Clinically Important Effect-Non-responder”

Responder area overlapped by CIm for Line 1 has “Possible” added to Responder label and area not overlapped has “Probable” added.

Nonresponder area overlapped by CIm for Line 2 has “Possible” added to Nonresponder label and area not overlapped has “Probable” added

1b2 CIm file. In this application a CIm is provided from an assessment done in a published research study. The 95% CIm for the mean of 3 MMSE assessments is +/−2.6 MMSE points

1b3 FDA approved drug CT outcomes and followup outcomes. This file provides the data to plot the outcomes from treatment in published studies of drugs for Alzheimer's disease treatment.

1c Working files, these are temporary files that hold the results of calculations

1c1 Calculated data for plots and calculations

2. Not used

3. Data Entry Routine

A subroutine to enter data from a web page into the appropriate patient record.

4. Not needed

5. Least Squares Line Fit

This is the statistical routine for fitting a line to a set of data by minimizing the sum or the squared differences of the fitted line points and the data

6. Confidence interval of measurement (CIm)

Draw from File 1b2

7. Plot data. A representative plot is as follows:

MMSE GRAPH

| MMSE Change | +4 | Provide indication for date of Medication Start, medication end, placebo start, placebo end | | |
|---|---|---|---|---|
| Score | +0 | | | |
| | −4 | | | |
| | −8 | | | |
| | 0 | 3 | 6 | 9 |
| | 0 | 3 Months | 6 Months | 9 Months etc. |
| | Jan 15 | April 15 | July 15 | and so forth |
| | 2002 | 2002 | 2002 | 2002 |

(Note that the months after starting medications and dates are obtained from file 1a1)

8. Plot line This is a routine for plotting any line on the graph

9. Plot CIm This is the routine for plotting the Cims on the graph

It show the values of a line as the line plot + and − the CIm (CIm could be plotted as outlying dotted lines or color shading in the area. For example, for patient data as a 5 Least squares line fit and 8 Line plot we want to convey that the patient's true clinical course is neither the points nor the least square line plot but the range within the +/−CIm. Thus for an expected course predicted from an earlier patient course measures fall outside the CIm range consistently in one direction we can support a ‘probable’ change in course and if they remain inside ‘probable no change.’ A CIm can also be shown around other lines for example a ‘Criteria of Clinical Significance’ or a “Mean of treated patient courses” each + and − the CIm

10. Calculate Means of three MMSE assessments

To calculate a mean of three MMSE assessments take three consecutive MMSE scores and average the MMSE scores. Record this average as the MMSE “Mean of three scores” for t=mean date of MMSE assessments. To find the t=0 MMSE for the graph take the three MMSE scores on or immediately prior to the date of starting medication and adjust the MMSE value to 0 for time t=0. Label and calculate other MMSE Means of 3 assessments in relation to this t=0 MMSE adjustment thus showing the change score. Do this as follows—

Take then the first three MMSE scores after starting medication and average the MMSE scores. Record this average as the MMSE “Mean of three scores” for t=1 where the date of t=1 is the mean of the dates of the three scores that were averaged. Now for t=n+1 take the next three MMSE scores after the scores used for t=n and average the MMSE scores. Record this average as the MMSE “Mean of three scores” for t=n+1 where the date of t=n+1 is the mean of the dates of the three scores that were averaged. Continue until no group of three MMSE scores is available. Plot as ‘means.’

If there are unused MMSE assessments prior to the three prior to start of medication calculate the MMSE mean score and time for these using the above iteration in reverse.

11. Not used

12. Not used

13. Not used

14. Not used

15. Define ‘Areas’ on Graph from file 1b1 and color using 16

16. Name and color patient course by ‘Area’

Areas are defined in file 1b1. These areas are defined as follows

Line 1 labeled “Estimated Untreated Alzheimer MMSE Loss” This line starts at t=0, MMSE=0 and goes to 6 months with MMSE=−2.0 at 12 months −4.0 and so forth. Extend line over remainder of graph x axis dimension

Line 2 labeled ‘Criteria of Clinical Significance’ this line starts at t=0, MMSE=0 and goes at 6 months with MMSE=−1.0, at 12 months −2 and so forth.. Extend line over remainder of graph x axis dimension

Lines 3 through 6− CIm s for first and second lines. Show CIm lines at +/− CIm in relation to line 1 and 2 and associate with shading. May be best as shaded colored areas that allow overlaps to be distinguished 2

Area above the Line 2 is labeled “Clinically Important Effect-Responder” and area below Line 2 “No Clinically Important Effect-Non-responder”

Responder area overlapped by CIm for Line 1 has “Possible” added to Responder label and area not overlapped has “Probable” added

Nonresponder area overlapped by CIm for Line 2 has “Possible” added to Nonresponder label and area not overlapped has “Probable” added

Color the areas with distinguishing colors.

17. Adjust Data Points

Use from 5 LSLF the Least Squares Y intercept (Y intercept is a in y=a+bx) a as follows.

First set a=0 to plot the least squares line (that is y=bx)

Second subtract a from each MMSE score in 1c 1(the calculated change scores for the patient's MMSE scores) to create 1c1 (A) Adjusted Data for Plots.

18. Change Score Conversion

To convert the 1a 1 Patient data MMSE scores into change scores proceed as follows

Using “Mean of 3 MMSE Scores” the MMSE score for t=0 or before is the last “mean of 3 MMSE scores” with an averaged date of the 3 scores before medication start. Consider this as MMSE (t=0) for calculations but plot at time t. T=0 is the time of medication start. To calculate change scores-calculate change in the MMSE score from the MMSE score at t=0

(Note t=0 as Medication Start on graph. The patient may have a clinical course prior to the t=0 MMSE assessments were done prior to the t=0 Mean of 3 assessments).

The construction of a Management Program for AD involves three types of pages: pages with narrative for the users information and instruction; pages for data entry and pages for presentation of results from analyses and interpretations. The flow of Management Sequencing is either programmed into the page presentations to the users or presented as options to be selected by clicking a button on the web page screen. The AD Management Sequences are provided to the user in FIG. VIII through XIX.

In these figures the patient data, whether the default data for the patient example used in the figures, Douglas Default, or data for a patient provided by the user, goes to 1a1 patient file where it is maintained with date. In this 1a1 file treatment dates and names of treatment are maintained as are records of dates when each Management Sequence is used with a patient. Thus an analysis can call on this file for required data about the patient's earlier management. This procedure is followed for all data entry and no special notes about following this procedure are provided for later data entry pages. Analysis and interpretation here and used elsewhere always goes to the following page for analysis and interpretation.

In FIG. VIII the data in Boxes 108 through 112 is entered into the patient file 1a1. In FIG. IX the patient data from file 1a1 is processed as in Table I to provide graph #1 Box 114.

TABLE I
For (name of patient) to provide Graph with characteristics #1
Go to Patient's record (1a1) and obtain MMSE records entered in Boxes 108-112 by date

And the date of initiating treatment
Go to
10 calculate means and standard deviations of three assessments. Following

instructions to calculate means of three assessments and enter data in file 1c1 (RM) Working
file (note R is for Raw and M for Means of three assessments.)

Go to
18 Change score conversion on data in 1c1 (RM) Working file then enter data

temporarily in a 1c1(RM-CS) Working file (Note CS is for Change score data)
Then on the data in 1c1(RM-CS) perform line fit-5 Least Squares line fit
Then using the results from 5 Least Squares line fit proceed to 17 Adjust data points in
1c1(RM-CS) Working file to create new working file 1c1(A) Working file (A for Adjusted
data)

7 Plot data points from 1c1(A)Working file
8 Plot line from 5 Least Squares line fit (it should originate at the 0 point on y axis

since this is change score plot) Call this line “Patient's Clinical Course”

Go to file 1b2 “CIm file” and obtain “Default CIm for mean of three MMSE tests”
Plot two lines to enclose the “95% CIm for Patient's Clinical Course” These lines

are Patient's clinical course + CIm and Patient's clinical course − CIm.
Highlight any MMSE values outside the “95% +/− CIm for Patient's Clinical Course”
Then Overlay “Clinically important response overlay” as follows
Acquire data from 1b1 “Clinically important response overlay”
15 Define ‘Areas’ of figure and color and name
For the last date for which there is a patient MMSE evaluation determine the value of the
MMSE from the least squares line fit “Patient's Clinical course”

Then determine in which area of the “Clinically important response overlay” this value falls and from that categorize the patient as one of the following according to the area containing the last score:

Probable responder

Possible responder

Possible non-responder

Probable non-responder

Call this “Patient outcome” and insert in narrative as described as follows: In Box 116 in place of “has a clinically important effect” and “probable” and “one chance in 20” shown for Douglas Default insert the statements appropriate to the “patient outcome”:

| | | | |
|---|---|---|---|
| “Patient Outcome” | “has a clinically important | “probable” | “only one chance in 20” |
| | effect” | | |
| Probable responder | has a clinically important | probable | only one chance in 20 |
| | effect | | |
| Possible responder | may have a clinically | possible | only better than even odds but |
| | important effect | | greater than one chance in 20 |
| Possible | may not have a clinically | possible | only better than even odds but |
| non-responder | important effect | | greater than one chance in 20 |
| probable | does not have a clinically | probable | only one chance in 20 |
| non-responder | important effect | | |

In place of “continued treatment” insert the appropriate wording

| | |
|---|---|
| “Patient outcome” | “Continued treatment with the” |
| Probable responder | continued trealment with the |
| Possible responder | continued trealment with the |
| Possible non-responder | alternative treatments should be considered after further |
| | evaluation of the |
| Probable non-responder | alternative treatments should be considered after further |
| | evaluation of the |

In FIG. X the data from Boxes 120-128 are entered into the patient data file 1a1. In FIG. XI Graph #2 and comments are produced as described in Table II

TABLE II
For the name of the patient produce a graph with characteristics #2 as follows:
Go to Patient's record (1a1) and obtain MMSE records entered from Boxes 120-128 by date
and the date of initiating treatment

Go to
10 calculate means and standard deviations of three assessments. Following

instructions calculate means of three assessments and enter data in file 1c1 (RM) Working
file (note R is for Raw and M for Means of three assessments.)

Then perform
18 Change score conversion on data in1c1 (RM) Working file then enter data

temporarily in a

1c1(RM-CS) Working file (CS is for Change score data)

Edit data to exclude MMSE data prior to date of initiating drug treatment and to

exclude data after date of initiating drug treatment plus 12 months.(Note this provides a data
set for the first year of drug treatment) Call this “First treatment year patient course” Then
on the “First treatment year patient course” data in 1c1(RM-CS) perform line fit-5 Least
Squares line fit

Then using the results from 5 Least Squares line fit to “First treatment year patient

course” proceed to 17 Adjust data points in all data in 1c1(RM-CS) Working file to create
new working file 1c1(A) Working file (A is for Adjusted data)

7 Plot data points from 1c1(A) Working file
8 Plot “First treatment year patient course” line from 5 Least Squares line fit (it

should originate at the 0 point on y axis since this is change score plot and should be
extended to all later dates for which there is data since this data is compared to this projection
and its CIm) Call this line “Patient's First Year Clinical Course on Treatment”

Go to file 1b2 “CIm file” and obtain “Default CIm for mean of three MMSE tests”
Plot two lines to enclose the “95% CIm for Patient's Clinical Course” The lines are

Patient's clinical course + CIm and Patient's clinical course − CIm.
Highlight any MMSE values outside the “95% +/− CIm for Patient's Clinical Course”

In Box 132 if the last MMSE value falls

(i) Within the “95%+/−CIm for Patient's Clinical Course” then “no change has occurred” and “Continued treatment with current medication” become no change has occurred and Continued treatment with current medication respectively.

(ii) Above the “95%+/−CIm for Patient's Clinical Course” then “no change has occurred” and “Continued treatment with current medication” become improvement occurred and continued treat ment with current medication respectively.

(iii) Below the “95%+/−CIm for Patient's Clinical Course” then “no change has occurred” and “Continued treatment with current medication” become a deterioration has occurred and consideration of alternative dosing or treatment respectively.

In Boxes 134 and 136 the buttons take the user to the selection.

In FIG. XII the buttons in Box 138 take the reader to the selection. In FIG. XIII the data in Boxes 140 through 144 are entered into patient file 1a1. In FIG. XIV the graph #3un Box 146 is constructed as in Table III

TABLE III
For the patient the graph Box 146 is constructed as follows:

Go to Patient's record (1a1) and obtain all MMSE records by date and arrange

chronologically

Then use subroutine 10 calculate means and standard deviations of three assessments.

Following instructions calculate means of three assessments and enter data in file 1c1 (RM)
Working file (R is for Raw and M for Means of three assessments.).

Then using subroutine 18 Change score convert in1c1 (RM) Working file then enter

data temporarily in a 1c1(RM-CS) Working file (CS is for Change score data)

Then on the data in 1c1(RM-CS) identify data between “date of initiating treatment”

and “treatment change to placebo” and call this “Patient's Treatment Course” and file as
1c1(RM-CS-TP)

Then on the data in 1c1(RM-CS-TP) perform line fit-5 Least Squares line fit and call

the result “Patient's treatment course”

Then using the results from 5 Least Squares line fit“Patient's treatment course”

proceed to 17 Adjust data points in 1c1(RM-CS) Working file to create new working file
1c1(A) Working file (A for Adjusted data)

7 Plot data from change scores found in 1c1 ® Working file
8 Plot Line from 5 Least Squares Line “Patient's treatment course” (note that this line

is plotted beyond over range of dates for which there is data since the comparison of placebo
data is to the CIm around this line)

9 Plot 95% CIm for mean of three measures from file 1b2 CIm

If all of the MMSE values for dates after change ‘from treatment to placebo”

(i) fall within the “95%+/−CIm for Patient's Clinical Course” then Box 148“a,” Box 150 “continued,” Box 152 “the,” and Box 154 “loss” become no, consideration of alternates to, no, loss respectively.

(ii) fall above the “95%+/−CIm for Patient's Clinical Course” then Box 148“a,” Box 150 “continued,” Box 152 “the,” and Box 154 “loss” become no, consideration of alternates to, no, loss respectively.

(iii) fall below the “95%+/−CIm for Patient's Clinical Course” then Box 148“a,” Box 150 “continued,” Box 152 “the,” and Box 154 “loss” become a, continued, the, loss respectively.

In Box 156 the Buttons take the user to the indicated resource.

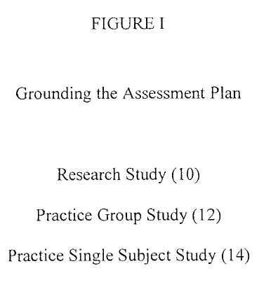

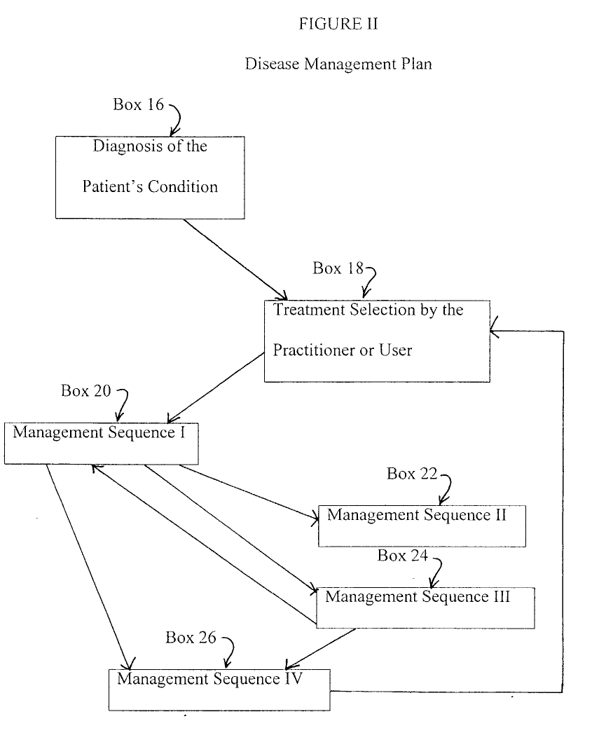

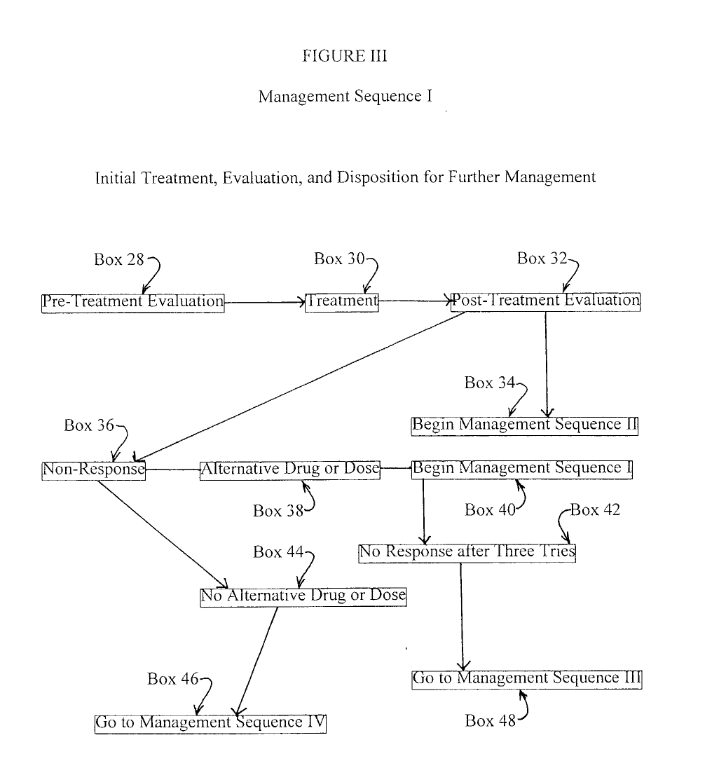

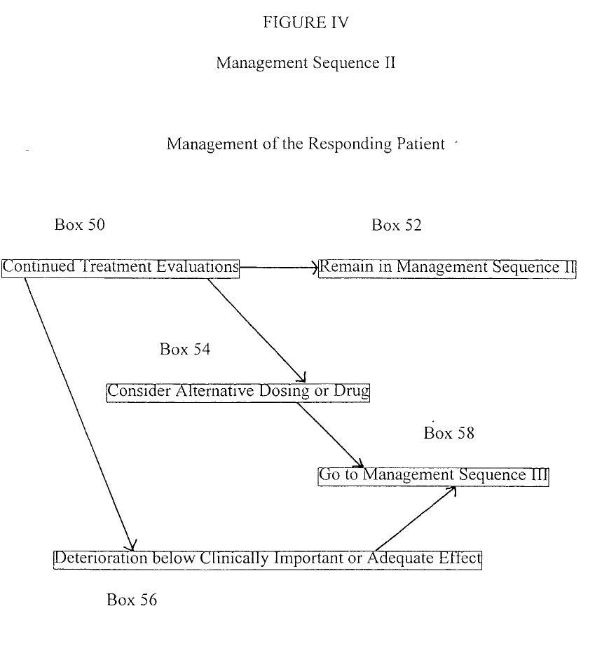

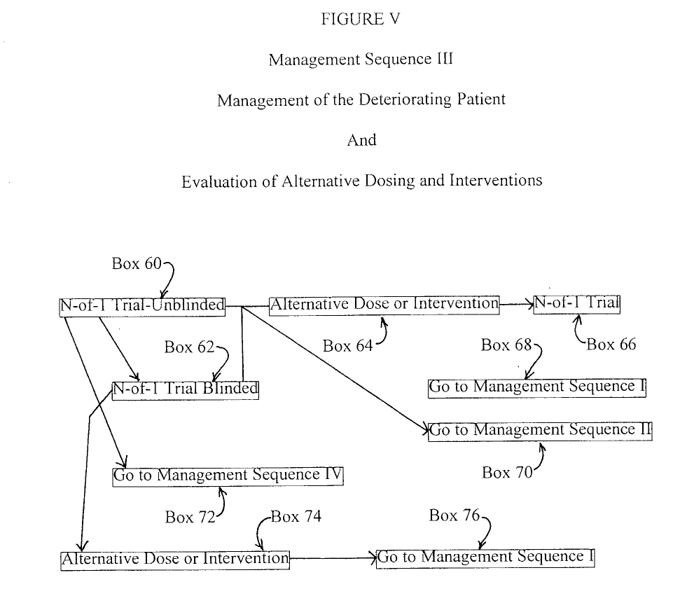

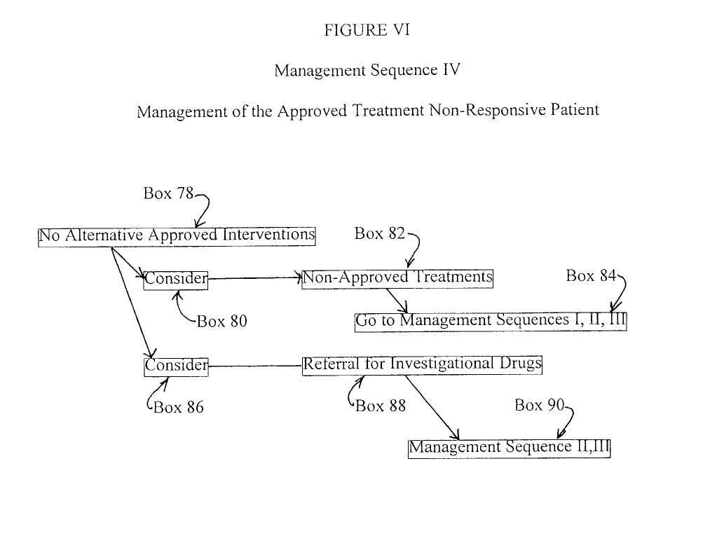

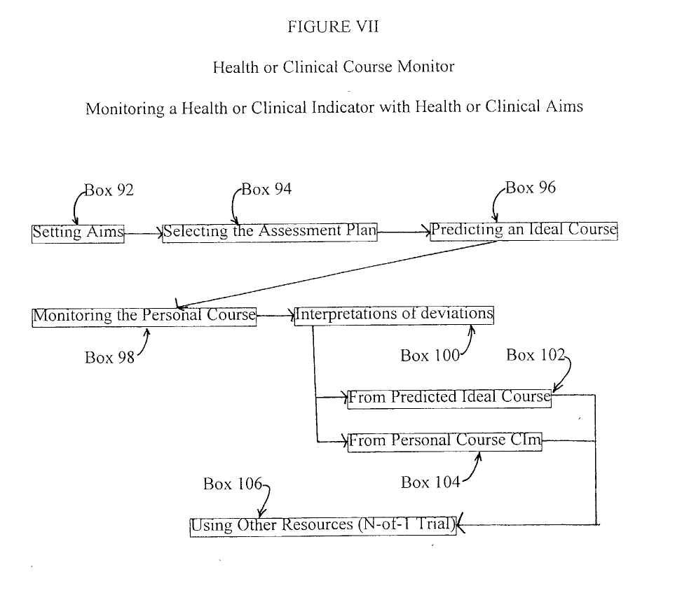

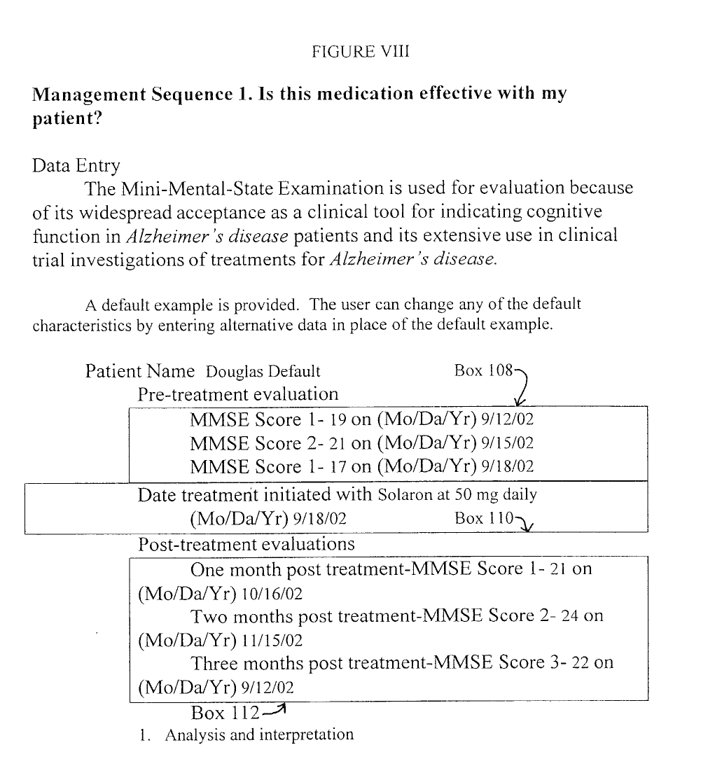

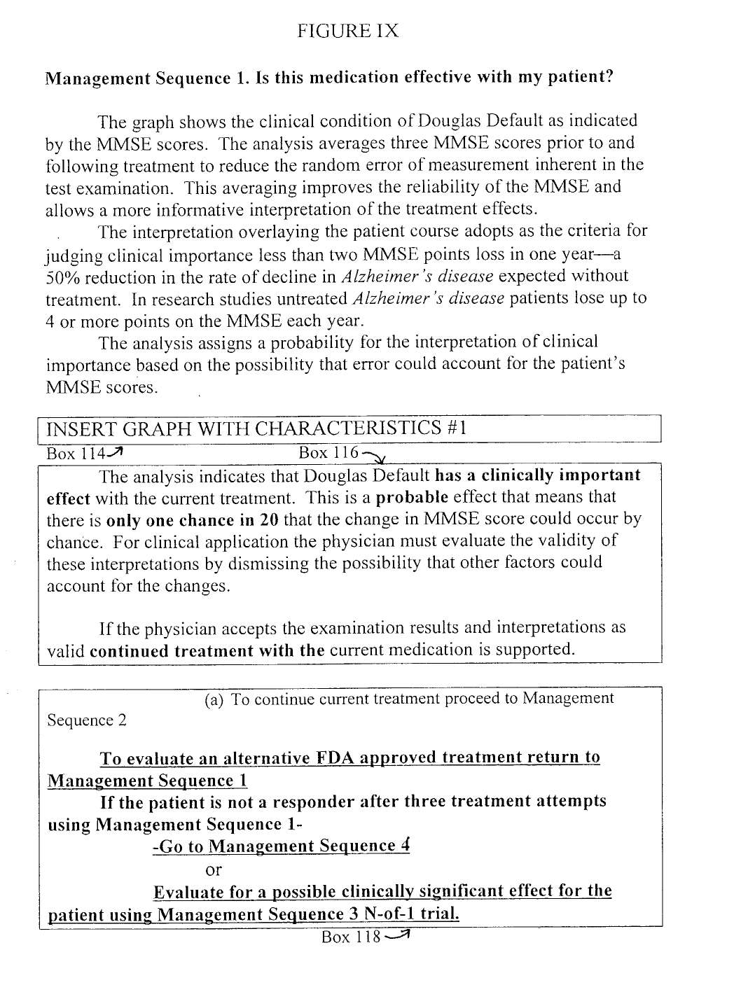

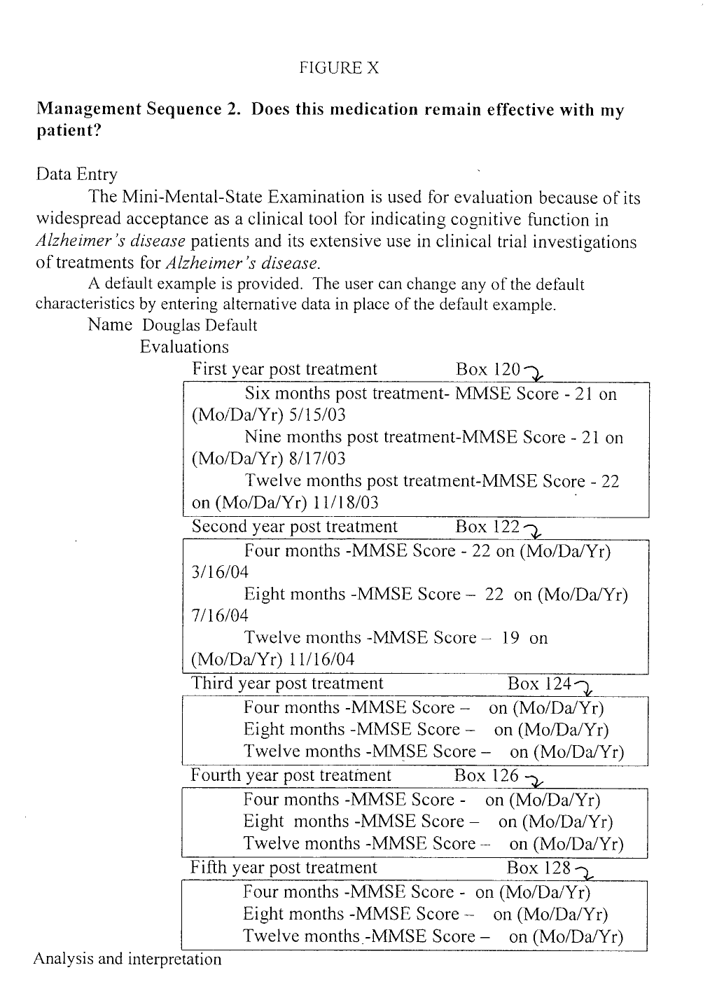

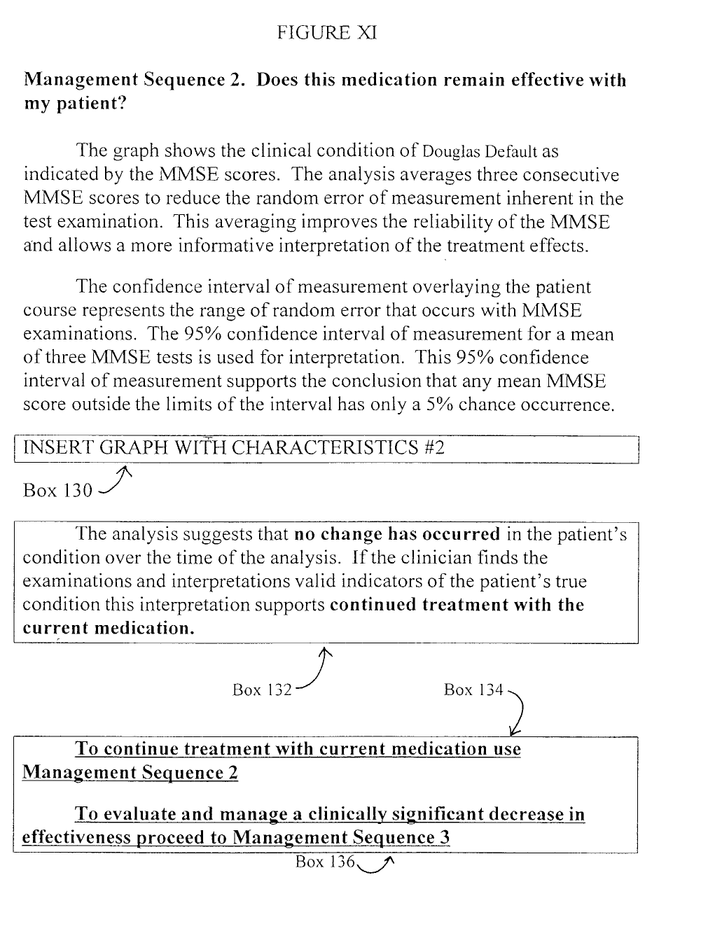

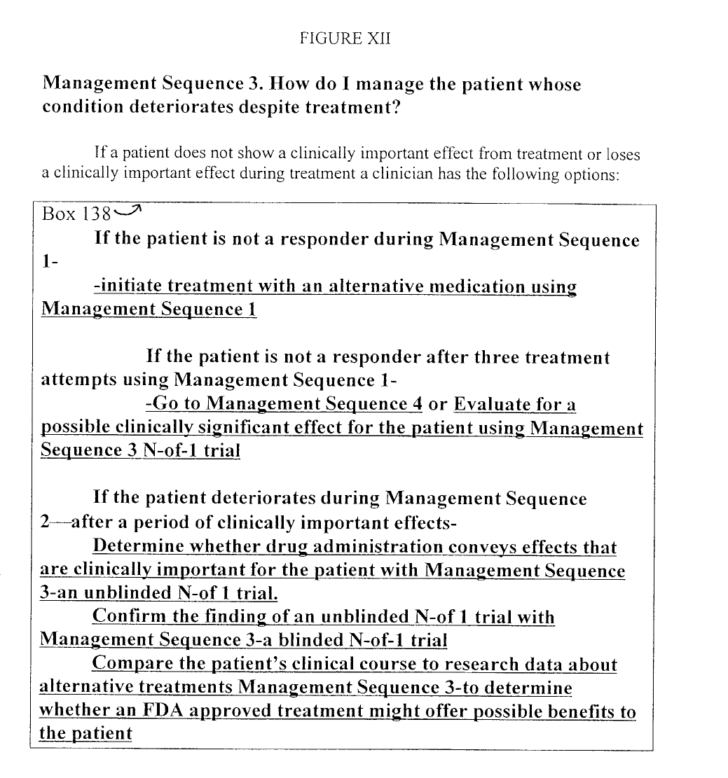

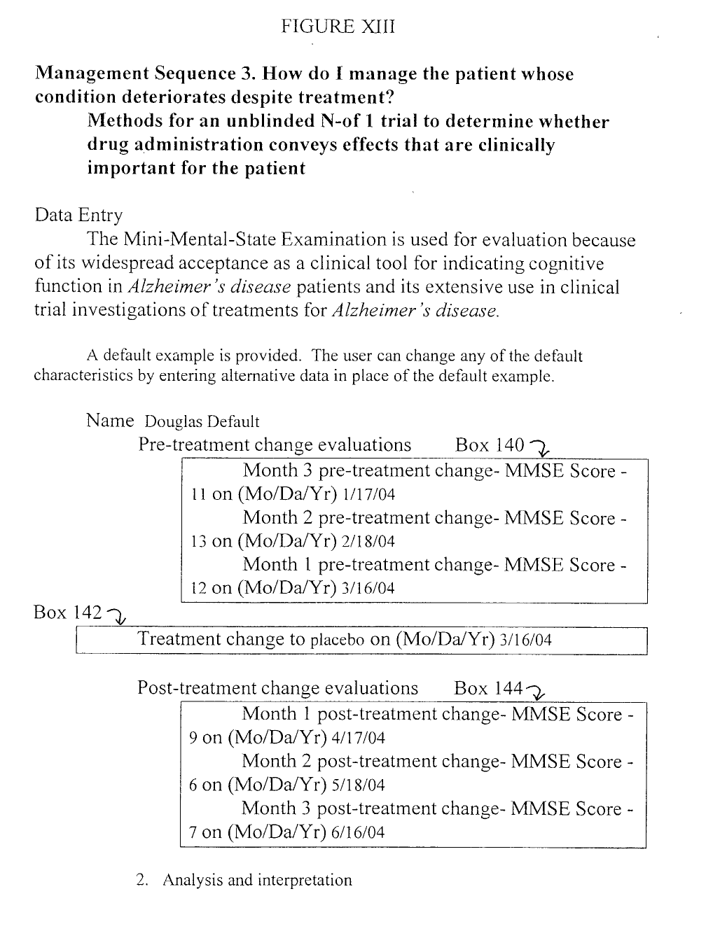

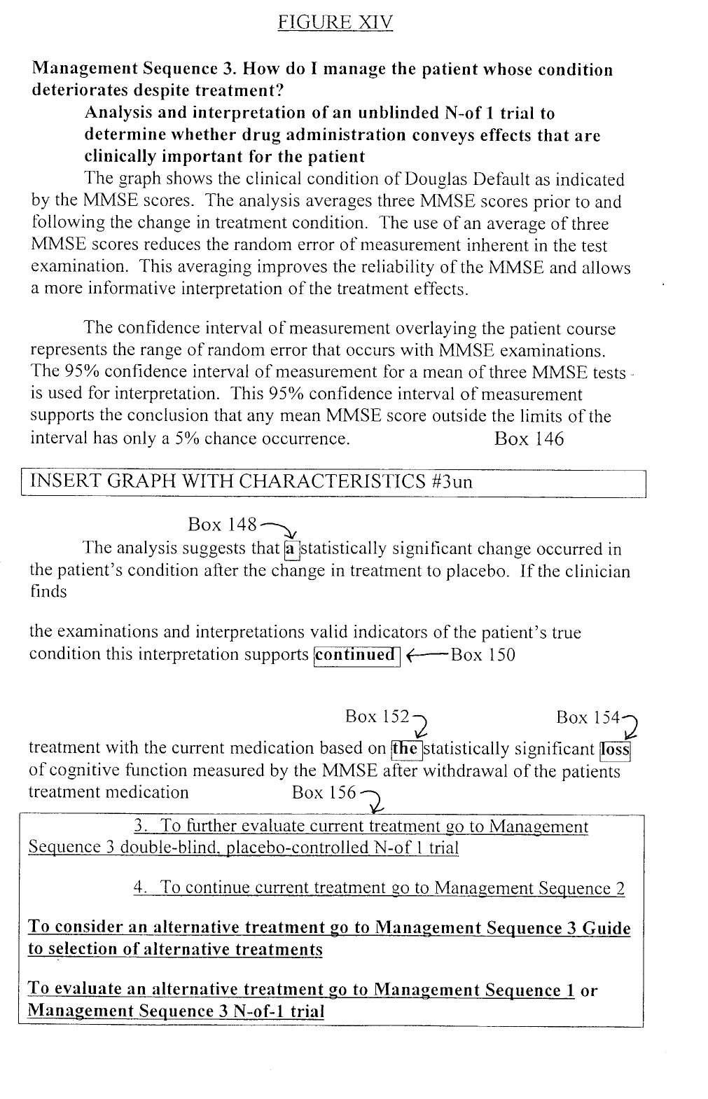

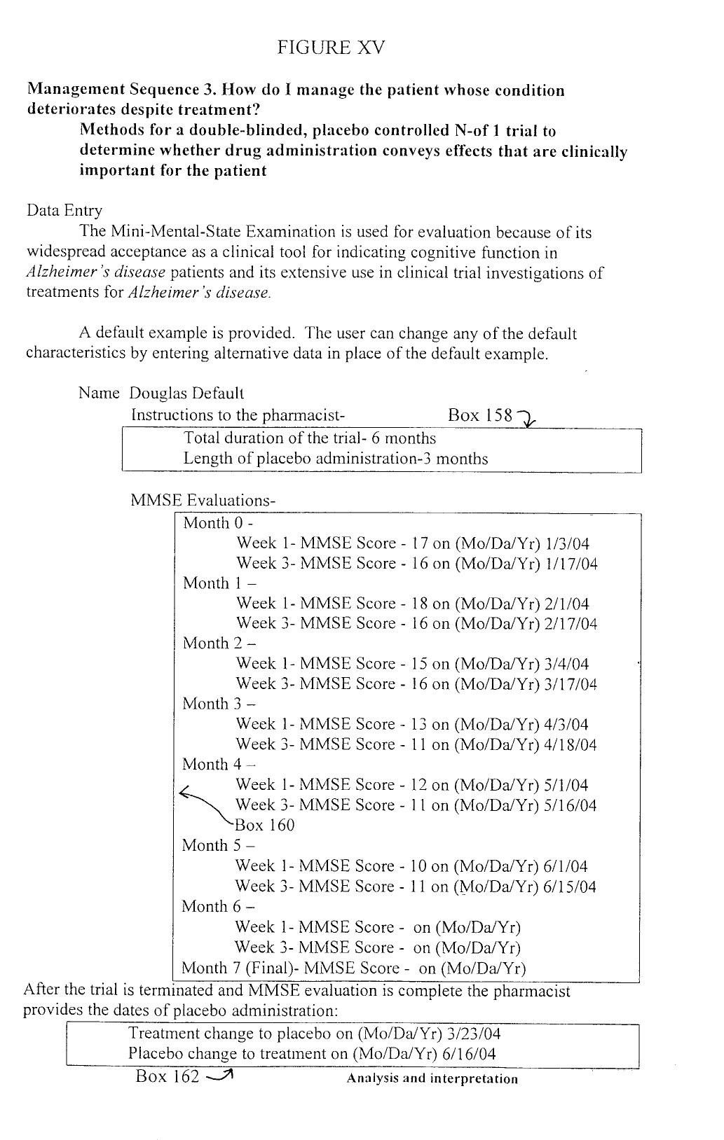

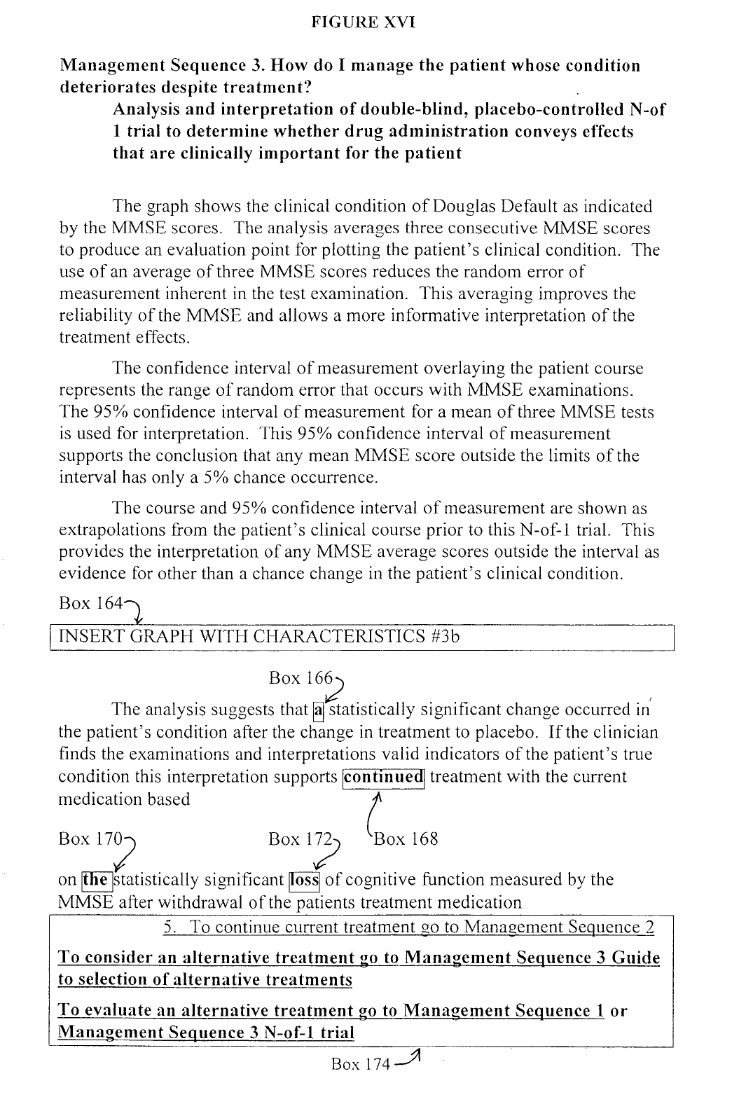

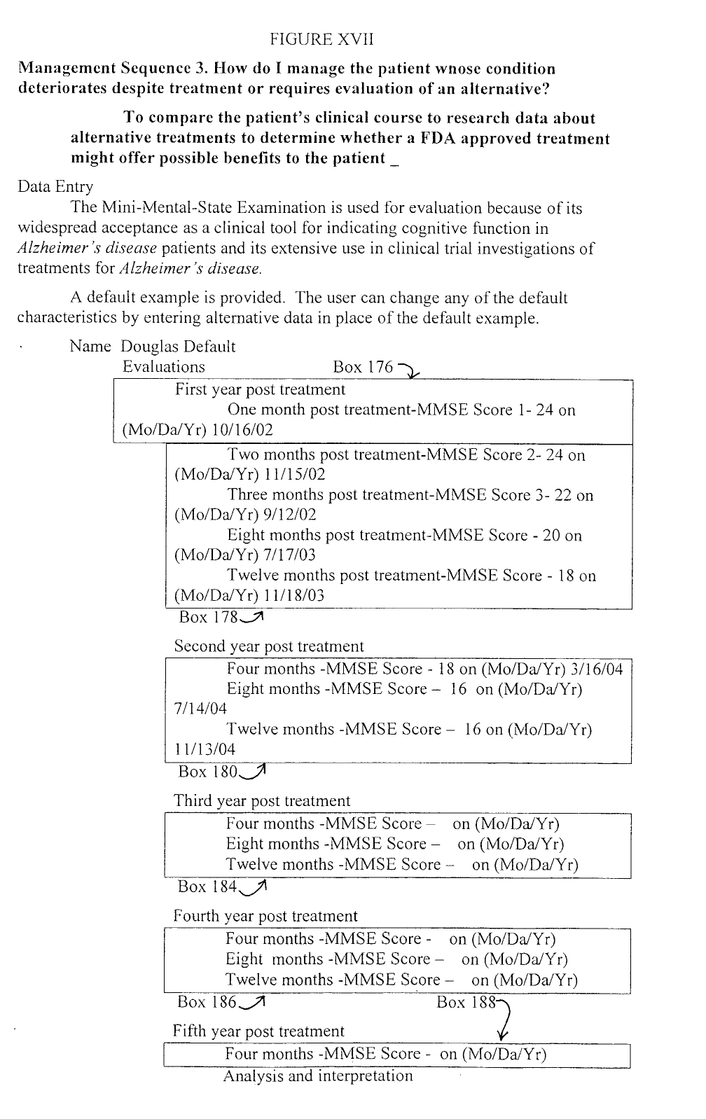

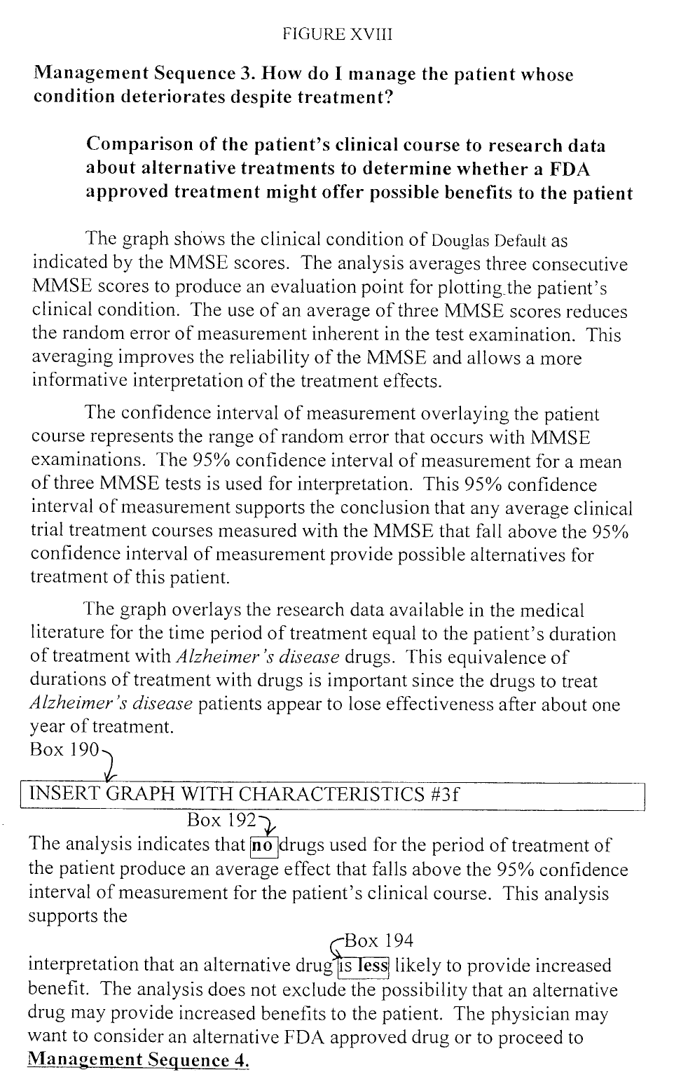

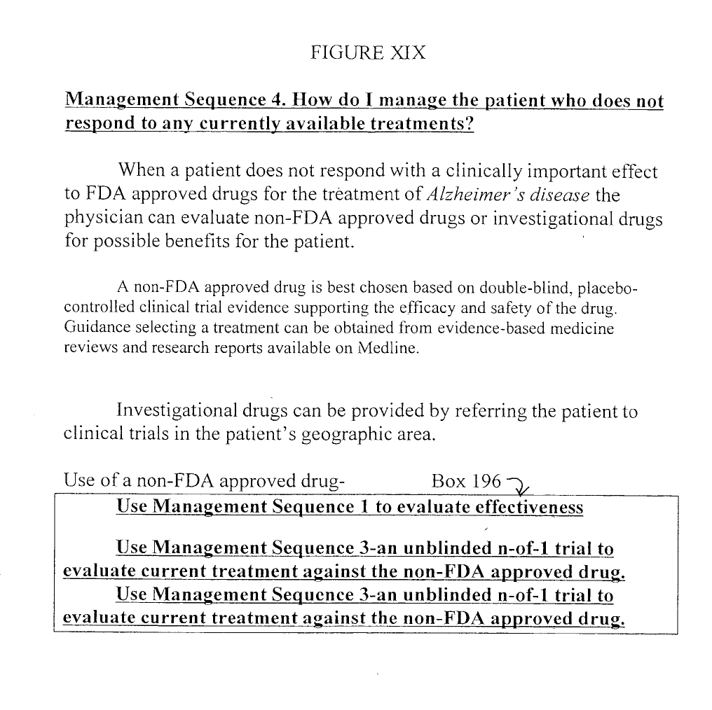

In FIG. 15 the data from Boxes 158-162 are entered in patient file 1a1. In FIG. XVI graph #3b Box 164 is constructed as described in Table IV

TABLE IV

For the patient construct graph #3b Box 164 by going to the patient record (1a1) to

obtain all MMSE records by date and arrange chronologically. Then use subroutine 10
calculate means and standard deviations of three assessments. Following instructions
calculate means of three assessments and enter data in file 1c1 (RM) Working file (note R is
for Raw and M for Means of three assessments.). Then with subroutine 18 Change score
conversion on data in 1c1 (RM) Working file then enter data temporarily in a 1c1 (RM-CS)
Working file (CS is for Change score data)

Then on the data in 1c1(RM-CS) identify data between “date of initiating treatment”

and “treatment change to placebo” and call this “Patient's Treatment Course” and file as
1c1(RM-CS-TP)

Then on the data in 1c1(RM-CS-TP) perform line fit-5 Least Squares line fit and call

“Patient's treatment course”

Then using the results from 5 Least Squares line fit“Patient's treatment course”

proceed to 17 Adjust data points in 1c1(RM-CS) Working file to create new working file
1c1(A) Working file (Note I call it A for Adjusted data)

7 Plot data from change scores found in 1c1 ® Working file
8 Plot Line from 5 Least Squares Line “Patient's treatment course” (note that this line

is plotted beyond over range of dates for which there is data since the comparison of placebo
data is to the CIm around this line)

9 Plot 95% CIm for mean of three measures from file 1b2 CIm
Then identify the MMSE values for dates after change ‘from treatment to placebo” but

before return to treatment if the placebo period has ended before the end of the trial. Then
use the same adjustments for Boxes 166-172 as taken for boxes 148-154 but using the data
from Graph #3b instead of #3un.

In FIG. XVII enter data into patient file from Boxes 176-188. In FIG. XVIII construct graph 3#f as described in Table V

TABLE V
For the patient construct graph with characteristics #3f as follows:
Go to Patient's record (1a1) and obtain MMSE records by date and the date of initiating
treatment

Go to subroutine 10 calculate means and standard deviations of three assessments.

Following instructions calculate means of three assessments and enter data in file 1c1 (RM)
Working file (note R is for Raw and M for Means of three assessments.) Use subroutine 18
Change score conversion on data in 1c1 (RM) Working file then enter data temporarily in a
1c1(RM-CS) Working file (Note CS is for Change score data)

Edit data to exclude MMSE data prior to date of initiating drug treatment and to

exclude data after date of initiating drug treatment plus 12 months.(Note this provides a data
set for the first year of drug treatment) Call this “First treatment year patient course”

Edit data to exclude MMSE data prior to date of initiating drug treatment plus 12

months.(Note this provides a data set for subsequent to the first year of drug treatment) Call
this “Second and following years of patient course”
Then on the “First treatment year patient course” data in 1c1(RM-CS) perform line fit- 5
Least Squares line fit
Then using the results from 5 Least Squares line fit to “First treatment year patient course”
proceed to 17 Adjust data points in all data in 1c1(RM-CS) Working file to create new
working file 1c1(A) Working file (A for Adjusted data)

7 Plot First treatment year patient course data points from 1c1(A)Working file
8 Plot “First treatment year patient course” line from 5 Least Squares line fit (it

should originate at the 0 point on y axis since this is change score plot and should be
extended to all later dates for which there is data since this data is compared to this projection
and its CIm) Call this line “Patient's First Year Clinical Course on Treatment”

Go to file 1b2 “CIm file” and obtain “Default CIm for mean of three MMSE tests”
Plot two lines to enclose the “95% CIm for Patient's First year Clinical Course”

These lines are Patient's first year clinical course + CIm and Patient's clinical course − CIm.
Then on the “Second and following years of patient course” data in 1c1(RM-CS) perform line
fit-5 Least Squares line fit
Then using the results from 5 Least Squares line fit to “Second and following years of patient
course” proceed to 17 Adjust data points in all data in 1c1(RM-CS) Working file to create
new working file 1c1(A) Working file (Note I call it A for Adjusted data)

7 Plot “Second and following years of patient course” data points from

1c1(A)Working file

8 Plot “Second and following years of patient course” line from 5 Least Squares line

fit (it should originate at the 12 months and should be extended to all later dates for which
there is data since this data is compared to this projection and its CIm) Call this line “Second
and following years of patient course on treatment”

Go to file 1b2 “CIm file” and obtain “Default CIm for mean of three MMSE tests”
Plot two lines to enclose the “95% CIm for Patient's Second and following years of

Clinical Course” These lines are Patient's clinical course + CIm and Patient's clinical course −
CIm.
Overlay Plots from file 1b3 FDA approved drug CT outcomes and followup outcomes

Then if no plots of CT outcomes fall above the CIm for the patient's course then in

Boxes 192-194 for “no” print no and for “less” print less. If one or more plots fall above the
CIm for the patient's course then for “no” print some and for “less” print a space (leave less
out).

In FIG. XIX Box 196 links take the user to the indicated resource.

Other analyses can also be provided in a Disease Management System. It is possible to estimate the probability that the drug or other health intervention is necessary to any change or lack of change of a person's condition by comparing the chance occurrence of each person's course as defined by the confidence interval of measurement for the outcome measurements to courses among actively and placebo treated persons or patients. It is also possible to determine, based on at least one long-term outcome of a patient's CIm defined clinical course whether the person's measured outcome will result in a long-term favorable outcome for the individual patient by comparison to data analyzed for long-term followup of persons with clinical courses that fall within one CIm of the patient's course. Similarly by identifying at least one optimal expected long term outcome, comparing a patient's expected long term outcome to the optimal expected long term outcome, and assessing the probability of whether the patient will achieve the optimal expected long term outcome useful information for judging the degree of current benefit can be gained. For this long-term followup of cohorts of patients with CIm defined clinical courses must be available.

One primary advantage of this identification of an error component and using the error component to define the error and true indicators of a patient's clinical course is the ability to compare a person's health or clinical course to the criteria of clinical significance to determine whether the person's indicated condition over time after an earlier assessment of treatment or intervention meets the aims of treatment for change or lack of change. Available options include:

(i) to compare a person's health or clinical course to the earlier course and confidence interval of measurement to determine whether the person's indicated condition continues to meet the aims of treatment for change or lack of change

(ii) to compare a person's health or clinical course and confidence interval of measurement to clinical courses of patients on alternative treatments or doses to determine whether a potentially more effective intervention for the person's indicated condition meets the aims of treatment for change or lack of change

(iii) to compare a person's health or clinical course in a blinded N-of-1 trial to the criteria of clinical significance to determine whether the person's indicated condition meets the aims of treatment for change or lack of change

(iv) to compare a person's health or clinical course in an unblinded N-of-1 trial to the criteria of clinical significance to determine whether the person's indicated condition meets the aims of treatment for change or lack of change

(v) to compare a person's health or clinical course in a blinded N-of-1 trial to the earlier and later clinical course and alternative treatment including placebo to determine the relative effectiveness of treatment conditions for the patient

(vi) to compare a person's health or clinical course in an unblinded N-of-1 trial to the earlier and later clinical course and alternative treatment including placebo to determine the relative effectiveness of treatment conditions for the patient

The method of invention organizes the resources of the method of invention into a Disease Management Plan specific for different diseases, treatments and purposes. It provide a Disease Management Plan comprised by at least one of the following Disease Management Sequences;

(i) Initial treatment evaluation and disposition

(ii) Continued treatment evaluation and disposition

(iii) Management of the patient with a deteriorating response to treatment

(iv) Management of the patient without clinically acceptable response to regulatory approved treatments or interventions

The method of invention provides for access to Disease Management Plans via a web-site and provides an Alzheimer's Disease Management Plan. It also allows for embodying any or all of the methods of invention in a health or symptom monitoring device or devices and integrating a health or symptom monitoring device or devices with a device that provides the methods of invention.

As may be recognized by those of ordinary skill in the pertinent art based on the teachings herein, numerous changes may be made to the above-described and other embodiments without departure from the spirit and scope of the invention as defined in the appended claims. Accordingly, this detailed description of preferred embodiments is to be taken in an illustrative, as opposed to a limiting sense.

I claim:

1. A method for assessing a person's response to a health intervention used to manage and treat a condition of the person, the method comprising the following steps:
identifying at least one outcome measure indicative of whether an individual patient's response to the health intervention meets an aim of treatment defined by a predetermined magnitude of change or lack thereof in the outcome measure;
defining an error component of the at least one outcome measure by performing at least one of the following to create a standard error of measurement:
(i) estimating error in the outcome measure by performing a test-retest of the outcome measure on a plurality of subjects and generating test-retest data on the outcome measure therefrom, generating a reliability statistic and standard deviation from the test-retest data, and calculating the standard error of measurement based on the reliability statistic and standard deviation;
(ii) estimating the error in the outcome measure by performing a test-retest of the outcome measure on a single subject and generating test-retest data on the outcome measure therefrom, generating from the test-retest data at least one of a standard deviation and a standard deviation adjusted for sample size, wherein the standard error of measurement is at least one of the standard deviation and the standard deviation adjusted for sample size;
selecting a criteria of statistical significance and a multiplier corresponding to the selected criteria of statistical significance;
generating an error component by multiplying the standard error of measurement by the multiplier to thereby ensure that any measurement with an outcome measure when the measurement falls outside of the error component will occur by chance with an average frequency not greater than the chance frequency defined with the criteria of statistical significance; and
using the error component to select the frequency of administration and summary statistic of the outcome measure in a manner that facilitates achieving the aim of treatment.

2. A method as defined in claim 1, wherein the step of estimating error in the outcome measure by performing a test-retest of the outcome measure is performed on a plurality of occasions on either a plurality of subjects or on a single subject.

3. A method as defined in claim 1, wherein the reliability statistics include at least one of a reliability coefficient, a generalizability coefficient, and a randomization statistic.

4. A method as defined in claim 1, wherein the error component is expressed as a confidence interval of measurement (“CIm”) or equivalent statistic.

5. A method as defined in claim 1, wherein the standard error of measurement is calculated based on data from a CT, other research, or clinical practice.

6. A method as defined in claim 1, wherein the frequency of administration and summary statistic of the outcome measure are selected such that the error component is less than the predetermined magnitude of change in the outcome measure.

7. A method as defined in claim 1, wherein the step of estimating the error in the outcome measure by performing a test-retest of the outcome measure on a single subject includes fitting a regression line to the test-retest data and subtracting the values predicted by the regression line from the test-retest data to substantially remove the effects of any trends over time on the test-reset data.

8. A method as defined in claim 1, wherein the health intervention is at least one of a drug, medical procedure, surgical procedure, behavioral pattern, and counseling, used to manage and treat a condition of the person when the person is a patient.

9. A method as defined in claim 1, wherein the at least one outcome measure defines a predetermined magnitude of change or lack thereof and offers adequately precise measurements for the outcome measure to be used as a best available indicator of whether an individual person's response to the health intervention meets the aims of treatment.

10. A method as defined in claim 1, wherein the multiplier expresses the cumulative probabilities in a distribution.

11. A method as defined in claim 1, further comprising the step of defining a measurement that falls outside of the error component by chance, with an average frequency not greater than the chance frequency defined with the criteria of statistical significance, as a true indicator component of measurement.

12. A method as defined in claim 11, further comprising the following steps:
defining a best available indicator of whether an individual person's response to the health intervention meets the aim of adequately precise measurement required by treatment by comparing different outcome measures, different frequencies of administration of different outcome measures, and different summary statistics of different outcome measures, to select at least one outcome measure, frequency of administration and summary statistic, based on at least one of the following;
(i) The smallest error component available;
(ii) the smallest ratio of error component to true indicator component;
(iii) an error component that is less than the change or lack of change from the person's health state addressed by the treatment to the health state required by the criteria of health or clinical significance;
(iv) a smallest ratio available of error component to the change or lack of change from the person's health state addressed by the treatment to the health state required by the criteria of health or clinical significance;
(v) a smallest ratio available of error component to true indicator component to the change or lack of change from the person's health state addressed by the treatment to the health state required by the criteria of health or clinical significance; and
(vi) a smallest ratio of the density of outcomes, whether predicted or known, within one error component of the criteria of statistical significance compared to the density of outcomes, whether predicted or known, outside of one error component at the criteria of statistical significance.

13. A method as defined in claim 12, further comprising the following steps: developing an assessment plan that uses the at least one selected outcome measure, frequency of administration and summary statistic for the at least one administration of the at least one outcome measure in a manner that makes the at least one best available outcome measure, frequency of administration, and summary statistic a best available and adequately precise indicator of the person's actual health status for the purposes of the aims of treatment.

14. A method as defined in claim 12, further comprising the following steps:
using the assessment plan and criteria of statistical significance and criteria of statistical significance to at least one of:
(i) develop a person's course over time out of the health and clinical states indicated by the at least one outcome measure and methods of administration and summary statistic selected;
(ii) compare a person's course of health and clinical status to the criteria of clinical significance to determine whether the person's indicated condition meets the aims of treatment for change or lack of change;
(iii) compare a person's course of health and clinical status to the criteria of health significance to determine whether the person's indicated condition meets the aims of health for change or lack of change;
(iv) estimate the probability that the drug or other health intervention is necessary to any change or lack of change of a person's condition by comparing the chance occurrence of each person's course as defined by the confidence interval of measurement for the outcome measurements to courses among other actively and placebo treated persons and patients;
(v) determine based on at least one long-term outcome of other actively and placebo treated persons whether the person's current measured outcomes will result in a long-term favorable outcome for said person;
(vi) identify at least one optimal expected long term outcome of actively and placebo treated persons, comparing a person's expected long term outcome to the optimal expected long term outcome, and assessing the probability of whether said person will achieve the optimal expected long term outcome;
(vii) compare a person's health or clinical course to the criteria of clinical significance to determine whether the person's indicated condition over time after an earlier assessment of treatment or intervention continues to meet the aims of treatment for change or lack of change;
(viii) compare a person's health or clinical course to an earlier course and confidence interval of measurement to determine whether the person's indicated condition continues to meet the aims of treatment for change or lack of change;
(ix) compare a person's health or clinical course and confidence interval of measurement to clinical courses of patients on alternative treatments or doses to determine whether a potentially more effective intervention for the person's indicated condition meets the aims of treatment for change or lack of change;
(x) compare a person's health or clinical course in a blinded N-of-1 trial to the criteria of clinical significance to determine whether the person's indicated condition meets the aims of treatment for change or lack of change;
(xi) compare a person's health or clinical course in an unblinded N-of-1 trial to the criteria of clinical significance to determine whether the person's indicated condition meets the aims of treatment for change or lack of change;
(xii) compare a person's health or clinical course in a blinded N-of-1 trial to an earlier and later clinical course and alternative treatment including placebo to determine the relative effectiveness of treatment conditions for the patient; and
(xiii) compare a person's health or clinical course in an unblinded N-of-1 trial to an earlier and later clinical course and alternative treatment including placebo to determine the relative effectiveness of treatment conditions for the patient.

15. A method as defined in claim 14, further comprising the following steps:
providing a disease management plan specific for at least one disease and treatment comprising at least one of the following disease management sequences:
(i) initial treatment evaluation and disposition;
(ii) continued treatment evaluation and disposition;
(iii) management of the patient with a deteriorating response to treatment or alternatives to current treatment; and
(iv) management of the patient without clinically acceptable response to regulatory approved treatments or interventions.

16. A method as defined in claim 15, further comprising the step of providing access to at least one disease management plan via a web site.

17. A method as defined in claim 15, wherein the at least one web-based disease management plan is an Alzheimer's disease management plan.

18. A method as defined in claim 1, wherein the frequency of administration and summary statistic of the outcome measure are selected based on adequately precise measurement expressed as at least one of the following:
(i) the smallest error component available;
(ii) the smallest ratio of error component to true indicator component;
(iii) an error component that is less than the change or lack of change from the person's health state addressed by the treatment to the health state required by the criteria of health or clinical significance;
(iv) the smallest ratio available of error component to the change or lack of change from the person's health state addressed by the treatment to the health state required by the criteria of health or clinical significance;
(v) the smallest ratio available of error component to true indicator component to the change or lack of change from the person's health state addressed by the treatment to the health state required by the criteria of health or clinical significance; and
(vi) the smallest ratio of the density of outcomes, whether predicted or known, within one error component of the criteria of statistical significance compared to the density of outcomes, whether predicted or known, outside of one error component at the criteria of statistical significance.

19. A method as defined in claim 1, wherein the step defining the error component includes determining the error of measurement of a single administration of an outcome measure and the error of measurement for multiple administrations of an outcome measure summarized as a summary statistic, and further including the step of evaluating the health status of a person based on the adequacy of measurement, the outcome measure, frequency of administration and summary statistic to be used to evaluate the health status of the person.

20. A method as defined in claim 13, wherein the step of developing an assessment plan includes identifying at least one outcome measure with a predetermined magnitude of change or lack thereof, wherein the outcome measure used with a frequency of administration and summary statistic expressing the results from administration offers adequately precise measurements for the outcome measure to be used as the best available indicator of whether an individual person's response to a health intervention meets the aims of treatment.

21. A method as defined in claim 18, wherein the step of defining the best available indicator of whether an individual person's response to a health intervention meets the aim of adequately precise measurement required by treatment includes comparing different outcome measures, different frequencies of administration of different outcome measures, and different summary statistics of different outcome measures, and selecting at least one outcome measure, frequency of administration and summary statistic based on the adequately precise measurement.

22. A method as defined in claim 1 wherein the step of using the error component of measurement, adequately precise measurement, assessment plan and criteria of statistical significance and criteria of clinical significance, characterize a person's course over time out of the health and clinical states indicated by the at least one outcome measure and methods of administration and summary statistic selected.

23. A method as defined in claim 1, further comprising the step of comparing a person's course of health and clinical status to the criteria of clinical significance to determine whether the person's indicated condition meets the aims of treatment for change or lack of change.

24. A method as defined in claim 1 further, comprising the step of comparing a person's course of health and clinical status to a criteria of health significance to determine whether the person's indicated condition meets the aims of health for change or lack of change.

25. A method as defined in claim 1, further comprising the step of estimating the probability that the health intervention is necessary to any change or lack of change of a person's condition by comparing the chance occurrence of each person's course as defined by a confidence interval of measurement for the outcome measurements to courses among other actively and placebo treated persons and patients.

26. A method as defined in claim 1, comprising the step of determining based on at least one long-term outcome of other actively and placebo treated persons whether the person's current measured outcomes will result in a long-term favorable outcome for said person

27. A method as defined in claim 1, further comprising the step of identifying at least one optimal expected long term outcome of actively and placebo treated persons, comparing a the person's expected long term outcome to the optimal expected long term outcome, and assessing the probability of whether said person will achieve the optimal expected long term outcome.

28. A method as defined in claim 1, comprising the step of comparing a person's indicated course to the criteria of clinical significance to determine whether the person's indicated condition over time after an earlier assessment of treatment or intervention continues to meet the aims of treatment for change or lack of change.

29. A method as defined in claim 1, further comprising the step of comparing a person's health or clinical course to an earlier course and confidence interval of measurement to determine whether the person's indicated condition continues to meet the aims of treatment for change or lack of change.

30. A method as defined in claim 1, further comprising the step of comparing a person's health or clinical course and confidence interval of measurement to clinical courses of patients on alternative treatments or doses to determine whether a potentially more effective intervention for the person's indicated condition meets the aims of treatment for change or lack of change.

31. A method as defined in claim 1, further comprising the step of comparing a person's health or clinical course in a blinded N-of-1 trial to the criteria of clinical significance to determine whether the person's indicated condition meets the aims of treatment for change or lack of change.

32. A method as defined in claim 1, further comprising the step of comparing a person's health or clinical course in an unblinded N-of-1 trial to the criteria of clinical significance to determine whether the person's indicated condition meets the aims of treatment for change or lack of change.

33. A method as defined in claim 1, further comprising the step of comparing a person's health or clinical course in a blinded N-of-1 trial to an earlier and later clinical course and alternative treatment including placebo to determine the relative effectiveness of treatment conditions for the person.

34. A method as defined in claim 1, further comprising the step of comparing a person's health or clinical course in an unblinded N-of-1 trial to an earlier and later clinical course and alternative treatment including placebo to determine the relative effectiveness of treatment conditions for the person.

35. A method as defined in claim 13, wherein the step of developing an assessment plan includes developing an assessment plan containing information concerning at least one of: (i) whether different outcome measures support the aims of intervention with adequately precise measurement; (ii) how outcome measures are combined into summary statistics to meet the aims of the intervention; (iii) how frequently outcome measures or combinations of outcome measure administrations needed to form summary statistics are administered to patients; (iv) how multiple administrations avoid carryover effects; (v) which single measure or summary statistic for multiple administrations is used in data analysis to control the error component of measurement to evaluate an intervention; and (vi) which single measure or summary statistic for multiple administrations is used in describing the individual person's course over time.

36. A method as defined in claim 13, wherein the step of developing an assessment plan includes providing an assessment plan for judging clinical response to the conditions of treatment and including planned evaluations for at least one of the following:
(i) initial treatment evaluation and disposition;
(ii) continued treatment evaluation and disposition;
(iii) management of the patient with a deteriorating response to treatment or alternatives to current treatment;
(iv) management of the patient without clinically acceptable response to regulatory approved treatments or interventions; and
(v) monitoring health and clinical indicators.

37. A method as defined in claim 14, further comprising the step of using the assessment plan and criteria of statistical significance and criteria of statistical significance to develop a disease management plan comprising at least one of the following management sequences:
(i) initial treatment, evaluation and disposition where after diagnosis and selection of a treatment or intervention a pre-treatment evaluation defined in an assessment plan is carried out with the person, the intervention begins, and a post-treatment evaluation is carried out with the person;
(ii) continued treatment, evaluation and disposition where after demonstration of a clinically important response regular evaluations defined in the assessment plan are carried out with the person;
(iii) management of the patient with a deteriorating response to treatment or alternatives to current treatment where at least one of the following are used:
(a) an N-of-1 trial to determine whether a clinically beneficial effect derives from administration of the intervention; and
(b) comparisons of the person's course to the courses of persons treated with alternatives including both different treatments and different doses to identify how likely an alternative could provide greater benefits to the person;
(iv) management of the person's treatment without clinically acceptable response to regulatory approved treatments or interventions where the resources of other management sequences are used to evaluate treatments not currently approved for use in the person's condition or investigational drugs or procedures; and
(v) management of a course defined over time by at least one health and clinical outcome measure where a confidence interval of measurement is used to predict the future course with error component such that any actual evaluations outside the projected range of error can be considered as probable true indicators of a change in the expected course.

38. A method as defined in claim 1, wherein the method embodies a disease management (“DM”) sequence is conducted in accordance with following steps:
(i) identifying the aims of the DM and the anticipated applications of the DM in patient care;
(ii) conducting a test-retest reliability study of at least one outcome measure to be used in the DM and determining the error component of measurement of the at least one outcome measure based thereon;
(iii) identifying proposed outcome measures of each patient's medical condition, and determining whether the proposed outcome measures have adequately precise measurement to meet the aims of the DM and the anticipated applications of the DM in patient care;
(iv) developing an assessment plan for the DM by selecting the frequency and summary statistic for measurement of each patient's medical condition based on an error component of measurement offering sufficiently precise measurement to meet the aims of the DM;
(v) identifying criteria of clinical significance for use in the DM and in applications of the DM in patient care;
(vi) selecting criteria of statistical significance to set the level of chance occurrence for use in interpreting comparisons in the DM;
(vii) assessing at least one patient with the DM in accordance with the assessment plan; and further comprising at least one of the following steps:
(a) comparing each patient's clinical course to the criteria of clinical significance, and determining whether the patient's condition is improving or not based thereon;
(b) comparing each patient's clinical course to the criteria of clinical significance, and determining whether the patient's condition is deteriorating or not based thereon;
(c) comparing each patient's clinical course to the criteria of clinical significance, and determining whether the patient's condition is unchanged or not based thereon;
(d) comparing each patient's clinical course to the course predicted from an earlier course of the patient and determining whether the patient's condition is improving or not based thereon;
(e) comparing each patient's clinical course to the course predicted from an earlier course of the patient and determining whether the patient's condition is deteriorating or not based thereon;
(f) comparing each patient's clinical course to the course predicted from an earlier course of the patient and determining whether the patient's condition is unchanged or not based thereon;
(g) evaluating each patient's clinical course in an N-of-1 trial;
(h) estimating the probability that the drug or other medical procedure is necessary for improvement of an individual patient's condition by comparing the chance occurrence of each individual patient's clinical course among active and placebo treated patients in the DM;
(i) determining based on at least one long-term outcome of the DM whether the measured improvement will result in a long-term favorable outcome for the individual patient; and
(j) identifying at least one optimal expected long term outcome, comparing a patient's expected long term outcome to the optimal expected long term outcome, and assessing the probability of whether the patient will achieve the optimal expected long term outcome.
